(19) United States
(12) Patent Application Publication
(10) Pub. No.: US 2010/0249947 A1
(43) Pub. Date: Sep. 30, 2010

(54) POROUS IMPLANT WITH EFFECTIVE EXTENSIBILITY AND METHODS OF FORMING AN IMPLANT

(75) Inventors: Michael D. Lesh, Mill Valley, CA (US); Stewart Kume, Emerald Hills, CA (US); Randy J. Kesten, Los Altos, CA (US); Leo Centeno, East Palo Alto, CA (US); Jeff Zalewski, San Francisco, CA (US)

Correspondence Address: KNOBBE MARTENS OLSON & BEAR LLP, 2040 MAIN STREET, FOURTEENTH FLOOR, IRVINE, CA, 92614, US

(73) Assignee: Evera Medical, Inc., Foster City, CA (US)

(21) Appl. No.: 12/413,240

(22) Filed: Mar. 27, 2009

(51) Int. Cl. A61F 2/02; B32B 37/00

(52) U.S. Cl. 623/23.74; 156/84

(57) ABSTRACT

The implant includes an outer layer of ePTFE which exhibits extensibility normally not associated with ePTFE. The ePTFE is reduced in length by deforming the fibrils between the nodes while maintaining the nodes in a substantially flat configuration. Various implant configurations that can include the outer layer described are also disclosed.

The invention is directed to an implant and methods of forming an implant. In particular, the present invention is directed to methods and devices for making an implant covered by a porous layer of material such as expanded PTFE (ePTFE).

### BACKGROUND

A problem which is encountered when using some porous materials such as ePTFE to cover an implant is that ePTFE can be relatively rigid and inelastic in one or more directions due to the inherent nature of many forms of ePTFE. As ePTFE is formed, numerous elongate fibrils are created which extend between nodes as the PTFE is stretched. The fibrils are often aligned with one another which provides high tensile strength when the ePTFE is tensioned in the direction of these fibrils. As such, the ePTFE has very little elasticity and is very stiff when tensioned in this direction. Consequently, a structure, such as an implant, which is covered with ePTFE may be somewhat stiff and may resist simple deformation due to this inelastic nature.

The invention is directed in some aspects to implants and methods of forming an implant which overcome some of the drawbacks of prior art implants and their methods of manufacture.

### SUMMARY OF THE INVENTION

The invention provides, in some embodiments, a method of forming an implant which is covered with a porous material which exhibits relatively inelastic properties in one or more directions such as ePTFE. The ePTFE is provided as a sheet of material having a plurality of fibrils extending between nodes.

The ePTFE is reduced in length by deforming the fibrils to shorten the distance between the nodes while maintaining the nodes on substantially the same plane to produce a material which can readily be made substantially flat. The ePTFE is reduced in length at a plurality of locations and is ideally reduced in a substantially uniform manner across the length of the ePTFE. The reduction in length occurs at a microscopic level with the fibrils buckling or deforming to accommodate the reduction in length. As a result, the distance between the nodes is decreased while maintaining the nodes in a substantially flat layer. The ePTFE may be reduced in length at least 5% and even up to at least 50% depending upon the particular application.

The sheet may also be stretched transversely to spread apart the fibrils before reducing the length. Stretching the sheet transversely may increase the amount of length reduction possible by spreading apart the fibrils so that the fibrils may undergo larger deformations and therefore a larger length reduction.

The invention is also directed to, in some embodiments, an implant having a length which has been reduced in accordance with the methods of the present invention. The implant will exhibit the ability to deform and/or elongate in a manner which is not possible with normal ePTFE while retaining the ability to create a smooth surface which is desirable in many implant applications. The smooth surface may, of course, include surface irregularities once implanted into the patient and such organic surface irregularities may be desirable.

Also disclosed herein is a method of forming an implant. First, provided is a layer of a polymer such as ePTFE and an inner element, the layer having a width and a length, the layer of ePTFE comprising a plurality of nodes and a plurality of fibrils extending between the nodes. Next, the length of the ePTFE layer is reduced while the ePTFE layer remains a substantially flat sheet. The layer of ePTFE can then be attached to the inner element after the reducing step. The reducing step can be carried out by reducing the length at a plurality of locations along the length of the ePTFE. The reducing step can reduce the length of ePTFE in a substantially uniform manner across the length of the ePTFE, or in a non-uniform manner. The fibrils between the nodes can deform under compression to accommodate the reduction in length. The reducing step can be carried out so that a distance between the nodes is decreased along the length while maintaining the nodes in a substantially flat layer. The length of the ePTFE layer can be reduced at least 5, 10, 15, 20, 25%, 50%, or more while maintaining a substantially smooth layer surface. The method can also include the step of stretching the layer of ePTFE to increase the width of the ePTFE layer before the reducing step, such as, for example, at least about 5, 10, 15, 20, 25%, 50%, or more. The providing step can be carried out with the inner element being a fluid permeable structure, e.g., open cell silicone, which permits fluid to pass therethrough.

Also disclosed herein is a method of forming an implant, that can include the steps of providing a layer of ePTFE and an inner element, the layer having a width and a length, the layer of ePTFE comprising a plurality of nodes and a plurality of fibrils extending between the nodes; and reducing the length of the ePTFE layer at least 10% while maintaining a substantially smooth surface of the layer. In some embodiments, the reducing step is carried out so that a distance between the nodes is decreased while maintaining the nodes in a substantially flat sheet. The fibrils between the nodes can be deformed to accommodate the reduction in length. The layer of ePTFE can be stretched to increase the width of the ePTFE layer before the reducing step. The layer of ePTFE can be attached to the inner element after the reducing step.

Also disclosed herein is an implant that includes a layer of ePTFE having a plurality of fibrils extending between nodes; and an inner element. The layer of ePTFE can be attached to the inner element. The layer of ePTFE can have a substantially flat surface. The ePTFE can have fibrils which are retained in a deformed, compressed configuration. In some embodiments, the layer of ePTFE has a length which has been reduced by deforming under compression the fibrils while maintaining the nodes within substantially the same plane. The layer of ePTFE can have a length that has been reduced by at least 5%, 10%, 25%, 50%, or more.

In another aspect, disclosed is a method of forming an implant, including the steps of providing a first layer of ePTFE, a second layer of ePTFE and an inner element, the first layer having a width and a length, the first and second layers of ePTFE comprising a plurality of nodes and a plurality of fibrils extending between the nodes; reducing the length of the first ePTFE layer while the first ePTFE layer remains a substantially flat sheet; and attaching the first and second layers of ePTFE to the inner element after the reducing step, the first layer being exposed to the exterior of the implant and the second layer being positioned between the first layer and the inner element. In some embodiments, the reducing step is carried out by reducing the length at a plurality of locations along the length of the first layer of ePTFE. The reducing step can reduce the length of ePTFE in a substantially uniform manner across the length of the ePTFE, or in a non-uniform manner. The fibrils between the nodes can deform under compression to accommodate the reduction in length. The reducing step can be carried out so that a distance between the nodes is decreased along the length while maintaining the nodes in a substantially flat layer. The length of the ePTFE layer can be reduced at least 5, 10, 15, 20, 25%, 50%, or more while maintaining a substantially smooth layer surface. The method can also include the step of stretching the layer of ePTFE to increase the width of the ePTFE layer before the reducing step, such as, for example, at least about 5, 10, 15, 20, 25%, 50%, or more. The method can also include the step of reducing a length of the second layer of ePTFE while the second ePTFE layer remains a substantially flat sheet before the attaching step. The attaching step can be carried out with the first layer being mounted over the second layer so that a first axis parallel to the length of the first layer is oriented within 30 degrees with a second axis parallel to the width of the second layer. The attaching step can be carried out with the first layer mounted over the second layer so that the length of the first layer is orientated substantially in the same direction as the width of the second layer.

Also disclosed herein is a method of forming an implant. The method can include the steps of: providing a first layer of ePTFE, a second layer of ePTFE and an inner element, the first and second layers having a width and a length, the first and second layers of ePTFE comprising a plurality of nodes and a plurality of fibrils extending between the nodes; reducing the length of the ePTFE layer at least 5% while maintaining a smooth surface. The reducing step can be carried out so that a distance between the nodes is decreased along the length while maintaining the nodes in a substantially flat layer. The fibrils between the nodes can be deformed to accommodate the reduction in length. The first layer of ePTFE can be stretched to increase the width of the first ePTFE layer. The first and second layers of ePTFE can be attached to the inner element after the reducing and stretching steps. The first layer can be exposed to native tissue, while the second layer is positioned between the first layer and the inner element.

Another aspect of the invention is an implant which may be implanted into a patient that includes a first layer of ePTFE having a plurality of fibrils extending between nodes, the first layer of ePTFE being exposed to contact native tissue when implanted into the body; a second layer of ePTFE also having a plurality of fibrils extending between nodes; and an inner element. The second layer of ePTFE can be coupled to the inner element. The first layer of ePTFE can have a reduced length while having a substantially flat surface. The ePTFE can have fibrils which are deformed to reduce a distance between the nodes. In some embodiments, the first layer of ePTFE has a length which has been reduced by deforming the fibrils while maintaining the nodes within substantially the same plane. The first layer of ePTFE can have a length which has been reduced by at least 5%, 10%, 25%, 50%, or more. The second layer of ePTFE can have a reduced length while having a substantially flat surface. The ePTFE can have fibrils which are deformed to reduce a distance between the nodes. In some embodiments, the second layer of ePTFE has a length which has been reduced by at least 5%, 10%, 25%, 50%, or more while retaining a substantially flat surface.

Also disclosed herein is an implant that includes a first layer of ePTFE having a plurality of fibrils extending between nodes. The first layer of ePTFE can be exposed to contact native tissue when implanted into the body. The implant can also include a second layer of ePTFE also having a plurality of fibrils extending between nodes; and an inner element. The second layer of ePTFE can be coupled to the inner element and be positioned between the first layer and the inner element. The first layer of ePTFE can have a reduced length while having a substantially flat surface, the ePTFE having fibrils which are deformed to reduce a distance between the nodes. The second layer of ePTFE can also have a reduced length while having a substantially flat surface, the ePTFE having fibrils which are deformed to reduce a distance between the nodes. In some embodiments, the first layer is mounted over the second layer so that the length of the first layer is oriented within no more than about 15, 30, or 45 degrees with the width of the second layer. The first layer can be mounted over the second layer so that the length of the first layer is oriented substantially with the width of the second layer. These and other aspects of the invention will become apparent from the following description of the preferred embodiments.

### BRIEF DESCRIPTION OF THE DRAWINGS

FIG. 1 shows an implant in accordance with the present invention.

FIG. 2 shows another implant in accordance with the present invention.

FIG. 3 shows a magnified view of the fibrils and nodes of a layer of ePTFE.

FIG. 4 shows a higher-power magnified view of the layer of FIG. 3.

FIG. 5 shows a magnified view of ePTFE before transverse stretching.

FIG. 6 shows the ePTFE of FIG. 5 after transverse stretching which spreads the fibrils apart and moves the nodes closer together.

FIG. 7 shows the sheet of ePTFE reduced in length in accordance with the present invention.

FIG. 8 shows a higher power magnified view of the layer of FIG. 7, illustrating the fibrils have been deformed to accommodate the reduction in length.

FIG. 9 schematically shows a sheet of ePTFE.

FIG. 10 schematically shows the sheet of ePTFE stretched transversely to increase the width of the sheet.

FIG. 11 schematically shows the sheet reduced in length in accordance with the present invention.

FIG. 12 is an exploded view of another implant having two layers of ePTFE on each side of the implant.

FIG. 13 is a schematic side elevational cross section through an empty sleeve in accordance with one embodiment of the present invention.

FIG. 14 is a side elevational cross sectional view through a partially inflated sleeve.

FIG. 15 is a side elevational cross sectional view through a filled sleeve having a uniform exterior profile.

FIG. 16 is a cross sectional side elevational view through a segmented sleeve, having customized fill volumes in each segment.

FIG. 17 is a cross sectional view through the distal end of an implant, illustrating a filler tube in position to fill a single segment.

FIG. 18 is a side elevational cross sectional view through a segmented sleeve having a plurality of internal baffles.

FIG. 19 is a side elevational schematic view of a filler tube in accordance with one embodiment of the present invention.

FIG. 20 is a side elevational view of an implant removably attached to a filler tube.

FIG. 21 is a side elevational schematic view of the implant of FIG. 20, positioned beneath the skin.

FIG. 22 is a side elevational view of an implant removably attached to a filler tube.

FIG. 23 is a side elevational schematic view of the implant of FIG. 22, positioned beneath the skin.

FIG. 24 is a side elevational schematic view of an implant and filler tube assembly, positioned within a delivery cannula.

FIG. 25A through 25D illustrate an assembly sequence for a soft tissue bulking device in accordance with one embodiment of the present invention.

FIG. 26 illustrates a bulking device as in FIG. 25D, additionally showing a guidewire.

FIG. 27 depicts non-limiting examples of potential locations for implants on the face.

FIG. 28 is an overhead plan view of a segmented malar mid-face implant in its deflated state, according to one embodiment of the invention.

FIG. 29 is a representation of a segment malar mid-face implant in its inflated state according to one embodiment of the invention.

FIGS. 30A-F are cross-sections of various preferred tube-based and sheet-based implants in various configurations. FIG. 30A illustrates the configuration of a tube-based, taut-filled implant. FIG. 30B shows a tube-based, slightly flaccid implant. FIG. 30C shows a tube-based markedly flaccid-filled implant. FIG. 30D shows a sheet-based, taut-filled implant. FIG. 30E discloses a sheet-based, flaccid filled implant. FIG. 30F shows an example of a sheet-based implant with two sheets of differing compliances that may be desirable in order to make an asymmetric cross-section upon inflation.

FIG. 31 illustrates various wrinkle lines of the face that may be treated with the disclosed implants, according to one embodiment of the invention.

FIG. 32 is an inflatable nasolabial implant in a deflated state, according to one embodiment of the invention.

FIG. 33A is a valve assembly, according to one embodiment of the invention.

FIG. 33B is an exaggerated cross-sectional view of the valve assembly of FIG. 33A.

FIG. 33C is a valve with a nitinol coil plug, according to one embodiment of the invention.

FIG. 33D is another valve with a nitinol coil plug in a different configuration, according to one embodiment of the invention.

FIG. 33E is a valve with a “paper clip” configuration of the nitinol coil plug, according to one embodiment of the invention.

FIG. 33F is another variation of a nitinol coil plug, according to one embodiment of the invention.

FIG. 33G is a valve with a folded O-ring configuration, according to one embodiment of the invention.

FIG. 34 is an inflatable nasolabial implant inflated to its maximally recommended fill volume, according to one embodiment of the invention.

FIG. 35A illustrates a cross-sectional view of a nasolabial implant fully inflated as recommended, near its distal end, according to one embodiment of the invention.

FIG. 35B illustrates an embodiment of an implant with variations in lamination, in which the porous outer material is affixed to the underlying elastomeric material in wound or interrupted configurations, for example, helical, bands, stripes, and the like.

FIG. 35C illustrates a cross-sectional view of a nasolabial implant inflated to less than the maximally recommended fill volume, near its distal end, according to one embodiment of the invention.

FIG. 35D is a view of a valve assembly within an embodiment of an inflated nasolabial implant, and illustrates the dual layer, bonded, and unbonded areas.

FIG. 36A is a cross-sectional view of a nasolabial implant fully inflated as recommended, near its proximal end according to one embodiment of the invention.

FIG. 36B is a cross-sectional view of a nasolabial implant inflated to less than the maximally recommended fill volume, near its proximal end according to one embodiment of the invention.

FIG. 37 shows a general shape of an advantageous nasolabial implant in which the distal end is to the left and the proximal end to the right according to one embodiment of the invention.

FIG. 38 shows an implantable prosthesis.

FIG. 39 is a top view of the implantable prosthesis of FIG. 38.

FIG. 40 shows an internal wall having an orifice.

FIG. 41 shows the internal wall in an expanded shape which reduces the size of the orifice.

FIG. 42 shows the internal wall separated from the rest of the prosthesis.

FIG. 43 shows the internal wall expanded to collapse the orifice.

FIG. 44 shows another implantable prosthesis having tension elements which may be selectively tensioned by the user.

FIG. 45 shows a plan view of the prosthesis of FIG. 44.

FIG. 46 shows another implantable prosthesis having tension members.

FIG. 47 shows a plan view of the prosthesis of FIG. 46.

FIG. 48 shows another implantable prosthesis having a chamber which may be filled or evacuated.

FIG. 49 shows the chamber of FIG. 48 expanded.

FIG. 50 is a plan view of the implantable prosthesis of FIG. 49.

FIG. 51 shows another implantable prosthesis.

FIG. 52 is a plan view of the implantable prosthesis of FIG. 51.

FIG. 53 shows an open cell structure.

FIG. 54 shows a membrane which is smaller than the open cell structure of FIG. 53.

FIG. 55 shows another implantable prosthesis.

FIG. 56 is a plan view of the implantable prosthesis of FIG. 55.

FIG. 57 shows still another implantable prosthesis having radially oriented channels.

FIG. 58 is a plan view of the implantable prosthesis of FIG. 57.

FIG. 59 shows another implantable prosthesis having circumferential channels.

FIG. 60 is a plan view of the implantable prosthesis of FIG. 59.

FIG. 61 shows still another implantable prosthesis with a circumferential channel.

FIG. 62 is a plan view of the implantable prosthesis of FIG. 61.

FIG. 63 shows another implantable prosthesis with a selective number of discrete attachments along the posterior and anterior walls.

FIG. 64 is a plan view of the prosthesis of FIG. 63.

FIG. 65 shows an implantable prosthesis which is attached to the membrane along two circular strips.

FIG. 66 is a plan view of the prosthesis of FIG. 65.

FIG. 67 shows a plurality of spacers positioned between the membrane and the open cell structure.

FIG. 68 shows another embodiment having a plurality of spacers.

### DETAILED DESCRIPTION OF THE PREFERRED EMBODIMENT

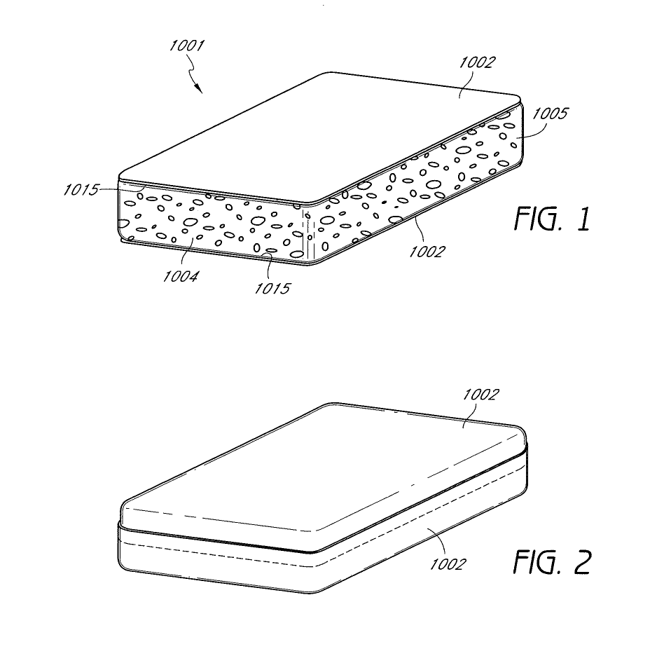

Referring to FIG. 1, disclosed is one embodiment of an implant 1001. The implant 1000 includes two layers of ePTFE 1002 which cover an inner element 1004. The ePTFE layer 1002 forms an outer layer which is exposed to native tissue. As such, the ePTFE layer 1002 provides desirable properties such as tissue ingrowth and biocompatibility. The implant 1001 may be shaped in any suitable manner depending upon the part of the body in which the implant 1001 is being used.

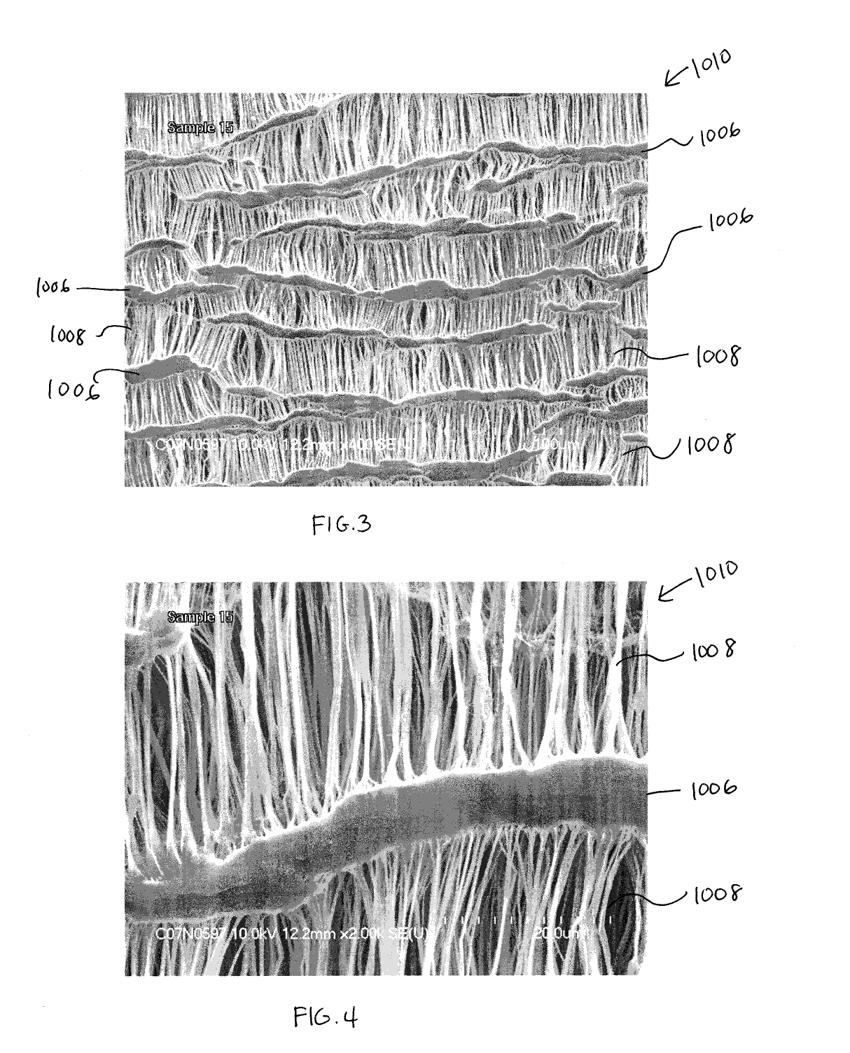

The inner element 1004 may be a porous and/or permeable material made of any suitable material such as an open cell silicone. Open cell structures and other features that can be used or modified for use with the implants and methods of use herein are described, for example, in U.S. patent application Ser. No. 12/024,835, filed Feb. 1, 2008 and hereby incorporated by reference in its entirety, particularly at FIGS. 1-31 and the accompanying description at paragraphs [0046] to [0061]. Furthermore, various features of implants described in U.S. Pat. Pub. No. 2006/0161253 published Jul. 20, 2006, the disclosure of which is hereby incorporated by reference in its entirety, can be used or modified for use with the implants and methods described herein, e.g., those described at FIGS. 1-25 and the accompanying text at paragraphs [0167] to [0272] of the publication. The inner element 1004 may also take many other forms such as a fluid or gel filled element or a soft or gel-like material, for example. FIG. 1 shows the inner element 1004 having exposed areas 1005 with one layer 1002 of ePTFE on opposing sides of the inner element 1004. The inner element 1004 may also be covered by any number of layers 1002 of ePTFE and in any suitable manner with or without exposed areas 1005 of the inner element 1004. For example, FIG. 2 shows the inner element 1004 completely covered by two layers 1002 of ePTFE, or at least one, two, three, four, five, or more layers in other embodiments.

Various forms of expanded PTFE, such as the layer 1002, generally has a structure such as that shown in the following FIGS. 3-8. The ePTFE material is created by expanding PTFE to break the PTFE into nodes 1006 connected by fibrils 1008. A sheet made of ePTFE is porous and the degree of porosity can be characterized by measuring the distance between nodes referred to as the Inter-Nodal Distance (IND) often characterized using an average IND.

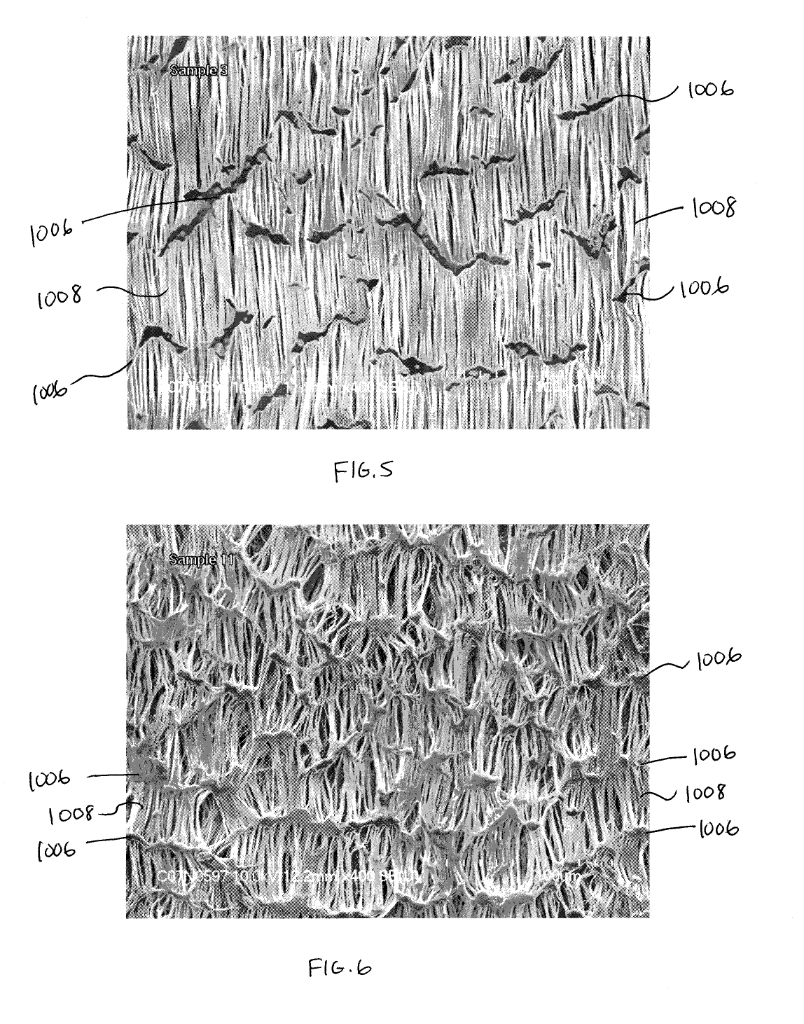

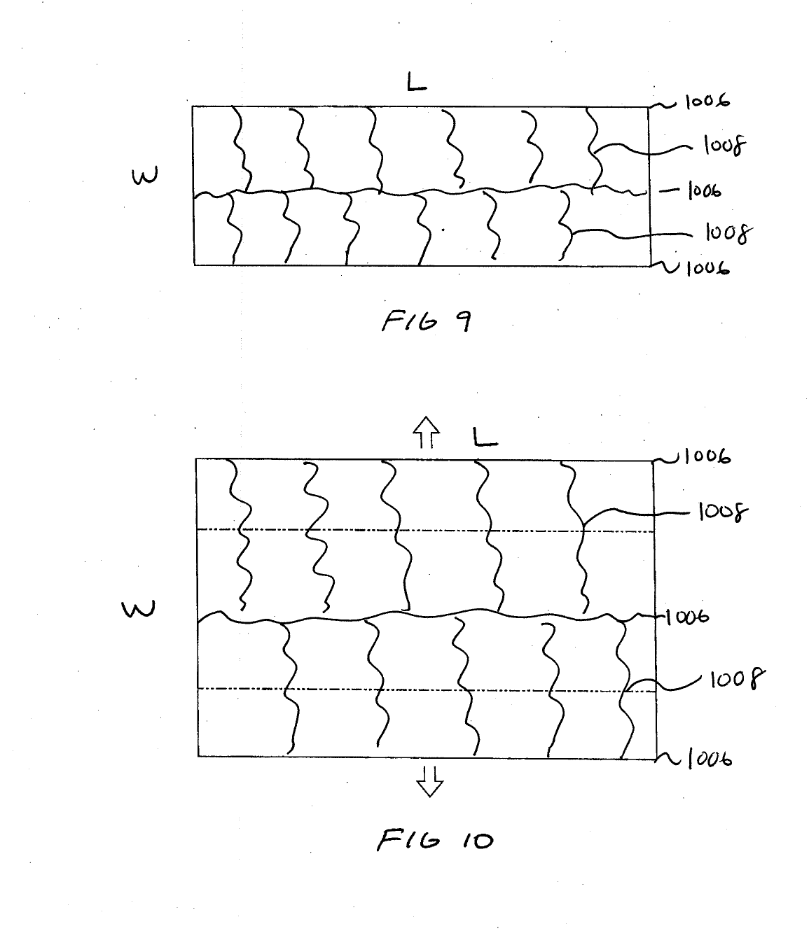

FIG. 3 illustrates a scanning electronic microscope (SEM) view of 0.150″ inside diameter ePTFE tubing that has already been radially expanded to a 0.250″ inside diameter, illustrating nodes 1006 connected by fibrils 1008. FIG. 4 is a higher power SEM view of FIG. 3. FIGS. 3-4 are prior to compression/shortening of the ePTFE as described herein that bring the nodes 1006 closer together. FIG. 7 illustrates an SEM view of the ePTFE layer of FIG. 3 after compression; FIG. 8 is a higher power SEM view of FIG. 4. As illustrated in FIGS. 7-8, the fibrils 1008 become deformed and non-linear as a result of the compression, and are curved or bent in one or more planes compared with the pre-compression fibrils of FIGS. 3-4.

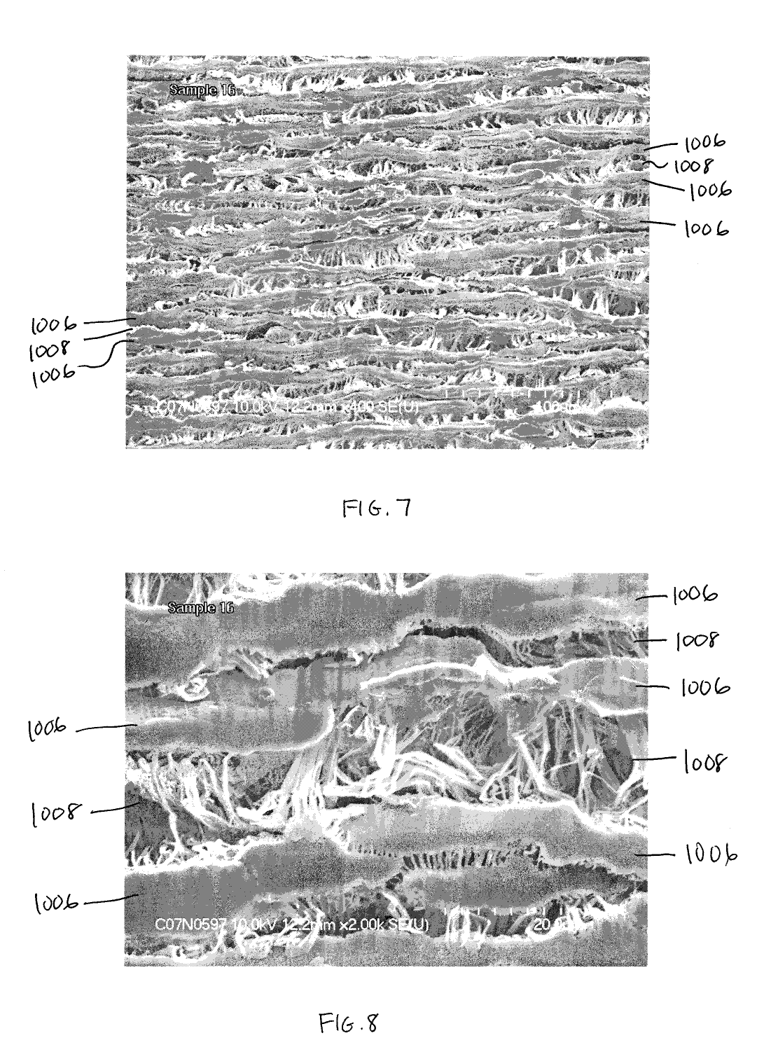

FIG. 5 is an SEM view of an ePTFE tubing layer prior to radial (transverse) stretching from a 0.150″ to a 0.250″ inside diameter, illustrating nodes 1006 connected by fibrils 1008. FIG. 6 illustrates the layer of FIG. 5 following radial stretching, which spreads the fibrils 1008 apart and moves the nodes 1006 closer together.

FIG. 9 schematically shows a sheet 1010 of ePTFE which has a length L and a width W. The terms length and width are used for orientation purposes only and may be used with reference to any regular or irregular shape and, furthermore, the width may be smaller, larger or equal to the length. The term “longitudinal” shall mean along the length L of the sheet while the term “transverse” shall mean along width W of the sheet.

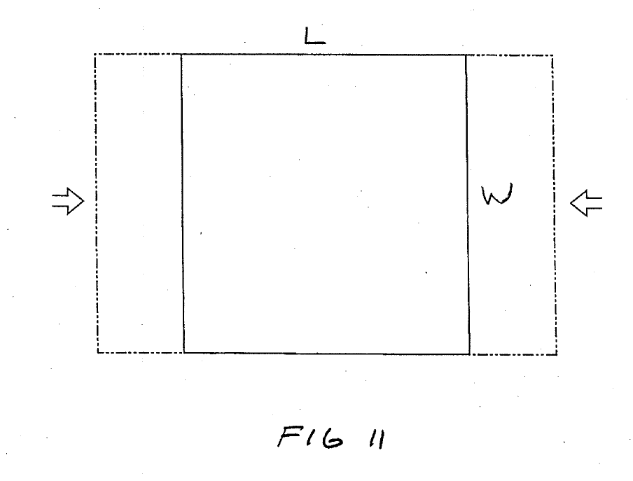

The sheet of ePTFE 1010 is oriented, in some embodiments, so that the fibrils 1008 extend generally longitudinally between the nodes 1006 as shown in FIGS. 9 and 10. Expanded PTFE may have anisotropic properties depending upon the manner in which it is formed. For example, the material shown in FIG. 3 has fibrils 1008 extending substantially parallel to one another in the direction in which the PTFE was expanded or stretched which is common to many forms of ePTFE. When the ePTFE is stretched or placed under tension longitudinally, the ePTFE has high strength and will undergo little elongation and exhibit virtually no elasticity. The high strength and low elongation in the lengthwise or longitudinal direction presents problems when using ePTFE since the inability to stretch or elongate in this direction may result in the implant resisting necessary deformation within the body.

A method of forming an implant 1001 is now described. Referring to FIGS. 9 and 10, the sheet 1010 of ePTFE may be first stretched transversely so that the fibrils 1008 are somewhat spread apart. FIG. 9 shows the sheet 1010 before transverse stretching and FIG. 10 represents an increase in the width from the dotted-line position after transverse stretching. FIG. 5 shows the fibrils 1008 before transverse stretching and FIG. 6 shows the fibrils 1008 spread apart after transverse stretching. The ePTFE may be stretched to increase the width at least 5%, at least 10%, at least 25% and even at least 50%, or more in the transverse direction, while being stretched to less than about 500%, 400%, 300%, 200%, or 100% of the unstretched transverse length in some embodiments. The ePTFE, in some embodiments, is preferably stretched to a point less than that of the rupture point of the material.

When the ePTFE is stretched in the transverse direction (e.g., as illustrated from FIG. 5 to FIG. 6), the ePTFE has relatively low strength and yields immediately to strain so that transverse deformation is plastic and cannot be recovered. Transverse stretching of the sheet 1010 may allow for greater cellular ingrowth as well as enabling a greater length reduction as described below. The ePTFE may be stretched by simply pulling on one or both sides of the sheet 1010. Alternatively, the ePTFE may be provided as a tube of material (not shown) which is stretched by increasing the diameter of the tube. The tube can then be cut to form the sheet 1010. Stretching the ePTFE transversely may be desirable in some applications but omitted in others without departing from various aspects of the present invention.

The length of the sheet 1010 of ePTFE is then reduced while maintaining the ability to present a substantially flat, smooth surface. The ePTFE is reduced in length by discretely reducing the length at numerous locations along the length with the goal to accomplish relatively uniform and distributed reduction in length. Of course, the sheet 1010 may have one or more discrete portions of the sheet 1010 having reduced length without departing from the scope of the invention. Care is taken during this process to not allow the ePTFE to wrinkle or otherwise form a macro-corrugation or fold since such features may be undesirable in certain cases.

The sheet 1010 of ePTFE is reduced in length in the following manner, according to some embodiments of the invention. The ePTFE is mounted to a smooth and lubricous surface, such as a sheet or rod of PTFE, HDPE or similar material (not shown). The reduction in length which is sought in accordance with the present invention is accomplished on a microscopic or relatively microscopic level as shown in FIGS. 7 and 8. The reduction in length is caused by deformation of the fibrils 1008 between the nodes 1006 rather than by forming macroscopic corrugations, wrinkles or folds that can be visualized, for example, by the naked eye. The fibrils 1008 are deformed by buckling, bending at a flexion point in one or more planes or curving in one or more planes all of which are desirable mechanisms for shortening the fibrils 1008 while maintaining a substantially flat surface free of macroscopic corrugations and folds. The nodes 1006 also shift slightly in the transverse direction and move toward one another longitudinally while remaining substantially in the same plane and in a relatively flat layer. The length L may be reduced at least 5%, 10%, 25% or even 50% or more while maintaining a substantially flat surface. Referring to FIG. 11, a graphic representation of the reduction in length is shown with the sheet 1010 being reduced in length from the dotted-line position to the solid line position while maintaining a substantially flat sheet or layer.

The reduction in length is not elastic and the sheet 1010 of ePTFE will maintain the reduced length if no forces are exerted on the sheet. The term “flat layer” or “substantially flat surface” shall mean that the ePTFE layer is substantially free of folds, corrugations and other macroscopic features and can readily be flattened. The sheet 1010 of ePTFE may, of course, include wrinkles due to the inherently flexible nature of the ePTFE sheet 1010 but the sheet 1010 reduced in length in accordance with the present invention can be readily made substantially flat and free of such features.

The reduction in length created in accordance with the present invention, in some embodiments is recoverable so that the ePTFE sheet 1010 may elongate to accommodate deformation of the implant which provides advantages over normal ePTFE which is generally inextensible in the direction of the fibrils 1008 (longitudinal). Unlike normal ePTFE, the present invention advantageously provides the sheet 1010 of ePTFE with a degree of extensibility due to the controlled reduction in length described herein.

Referring again to FIG. 1, the sheet 1010 of ePTFE is then attached to the inner element 1004 using any suitable method to create the layer 1002. For example, an adhesive 1015 may be used to attach the sheet 1010 to the inner element 1004. The layer 1002 may be intermittently attached to the inner element 1004 to permit the layer 1002 to recover the reduced length as necessary between unattached portions of the layer 1002. When the inner element 1004 is an open cell material, such as open cell silicone, the inherent structure of the open cell material may create sufficient space between attachments to the layer 1002 to permit the desired extensibility of the ePTFE. The inner element 1004 may also be elastic to also accommodate any change in length of the layer 1002. The requisite elasticity may even be provided by the adhesive 1015 when using an elastic material such as a silicone adhesive. The inner element can be attached to the outer layer through any appropriate fashion, such as bonding, stitching, or other methods as will be described, for example, below.

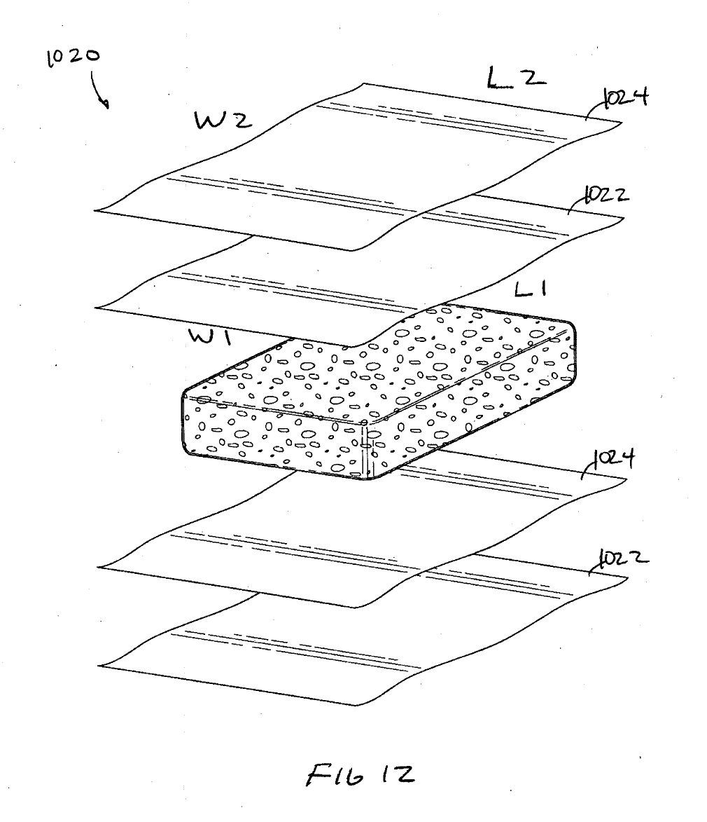

Referring to FIG. 12, an exploded view of another implant 1020 is shown. The implant 1020 includes a first layer 1022 of ePTFE and a second layer 1024 of ePTFE on each side of the implant 1020. The first layer 1022 of ePTFE has a length L1 which is reduced in accordance with the present invention and the second layer may also have a length L2 which has been reduced in accordance with the present invention. The second layer 1024 is oriented so that the fibrils 1008 (see FIGS. 3-8) of the second layer 1024 are substantially perpendicular to the fibrils 1008 of the first layer 1022. The two layers 1022, 1024 may be oriented in any other orientation, such as within 5, 10, 15, 20, 25, or 30 degrees of perpendicular, without departing from numerous aspects of the invention. The first layer 1022 and/or second layer 1024 may also be stretched to increase the width W1, W2 as described above and all discussion above concerning transverse stretching is incorporated here. In some embodiments it may be desirable to limit the amount of pre-stretching or transverse elongation so that some ability to stretch width-wise is retained.

The implant 1020 provides the same benefits as the implant 1001 described above in that the first and second layers 1022, 1024 of ePTFE may be stretched after implantation to provide a soft, pliable implant 1020. The first layer 1022 will be able to elongate lengthwise commensurate with the reduction in length undertaken in the manner described herein. When the first layer 1022 is stretched lengthwise, the second layer 1024 is stretched transversely. The first layer 1022 will elongate until the fibrils 1008 (see FIGS. 3-8) are substantially straightened at which time the first layer 1022 will greatly resist further elongation. In this manner, the first layer 1022 may help to limit excessive deformation of the second layer 1024 since the first layer 1022 will greatly resist elongation beyond the amount that the length was reduced prior to implantation. If the second layer 1024 has also undergone a reduction in length as described herein, the second layer 1024 may also elongate to the extent that the length was previously reduced at which time the second layer 1024 will then greatly resist further elongation. As such, the second layer 1024 may limit the amount that the first layer 1022 can increase in width.

### Additional Features and Implant Embodiments

The invention has been described in connection with certain embodiments, however, it is understood that numerous modifications may be made without departing from the invention. For example, the inner element 1004 may be a fluid filled element or may be a composite structure. Furthermore, the layer 1002 or sheet 1010 of material has been described as being ePTFE but may also be another suitable porous polymer, as, for example, described elsewhere in the application, which exhibits similar properties. Disclosed below are non-limiting embodiments of implant configurations that can have an outer layer, e.g., a porous polymer such as ePTFE with advantageous properties as described above, various inner elements as described below, along with various other features.

Some embodiments of the invention include a system and method for volume augmentation of tissue in a living being, preferably, a human. The system generally comprises a tissue-filling device and a method for delivering the tissue-filling device into tissue. The tissue-filling device comprises tissue filler material and an enclosing sheath. Preferably, the enclosing sheath forms a container that is filled.

The volume augmentation methods and devices described in embodiments of the present patent are intended to be used for tissue bulking in a variety of circumstances, depending on the need. For example: in gastroenterology, wherein increasing the volume of tissue at the gastro-esophageal junction can be used to treat gastro-esophageal reflux disease, and increasing the thickness of the gastric mucosa to decrease the volume of the stomach to treat morbid obesity; in urology, where placing filler radially around the urethra at the neck of the urinary bladder can ameliorate incontinence; and in cardiology, whereby tissue filler may be placed in the ventricular wall to decrease the volume of the left ventricular chamber to treat heart failure, or in the pericardial space to place pressure on the outside of the heart, also intended to decrease the volume of the heart chambers and thereby treat heart failure; and in other applications well known to those skilled in the art. In any of these clinical applications, the tissue-filling device may be combined with any number of other bioactive substances which may be released from the filler itself over time, or be injected concurrently.

One preferred use of the present invention is in the field of cosmetic plastic surgery wherein the system is used for augmentation in the dermis or subdermis to treat skin contour deficiencies caused by various conditions, including aging, environmental exposure, weight loss, child bearing, surgery, disease such as acne and cancer, or combinations thereof, or for beauty enhancement. The tissue augmentation method of preferred embodiments of the present invention is particularly suitable for treating frown lines, worry lines, wrinkles, crow's feet, facial scars, or marionette lines, or to augment facial features such as the lips, cheeks, chin, nose or under the eyes. Treatment of a patient may consist solely of using a tissue-filing device, or the tissue-filling device may be used as part of additional cosmetic surgery such as a face or brow lift. The characteristic of change from first configuration to second configuration makes the tissue-filling device desirable for use in endoscopic surgery. The tissue augmentation device may also be used for breast augmentation, and regions of the body that need volume enlargement during reconstructive plastic surgery, such as after trauma or tumor resection.

The sleeve can be embodied as a variety of structures, and constructed of a variety of materials. The term “sleeve” as used herein is meant to include any structure adapted to substantially separate a filler material from the tissue in which the tissue-filling device is implanted. The term “skin” and “membrane” is used interchangeably and has the same scope of meaning as sleeve.

In one embodiment, a sleeve is placed in the tissue to be filled, and as a second step, the sleeve is filled with material such that the sleeve, when filled, creates a volume adequate to alter the tissue contour as required to produce the clinical result. Filling can either be accomplished through the device used to implant the sleeve, or through a separate device, or both, as will be discussed. In an alternative embodiment, the tissue-filling device is constructed prior to its implantation in the tissue by filling a sleeve with a tissue filler and the assembled tissue-filling device is placed in the tissue. In still another alternative embodiment, the tissue filler may be of more than one component such that one (or more) component of the tissue filler is in place inside the sleeve before the sleeve is placed in the tissue to be augmented, and a second component (or components) are placed within the sleeve after the sleeve has been placed in the tissue, the combination of the components than constituting the final filler material.

The sleeve can be compliant or non-compliant, or a combination of compliant and non-compliant components. The sleeve may be made of a biocompatible but non-biodegradable material. Suitable materials include ePTFE, PTFE, polypropylene, polyacrylamide, polyurethane, silicone, polymethylmethacrolate, Dacron, metals, tubes or meshes of nickel titanium alloys such as Nitinol, silver, gold, platinum, or stainless steel. The sleeve can comprise a plurality of layers of materials. Other biocompatible materials are well known in the art, as, for example, disclosed in U.S. Pat. No. 5,630,844 to Dogan.

If fibrous tissue ingrowth is desired, then the sleeve can be made of or covered with ePTFE, as described above, with a pore size of in the range of from about 40 to about 100μ. If the filler material is, or becomes, non-flowable, the sleeve may be made of a biocompatible and biodegradable material chosen from any of various polylactides, polyglycolides, polycaprolactones, polyanhydrides, polyamides, polyurethanes, polyesteramides, polyorthoesters, polydioxanones, polyacetals, polyketals, polycarbonates, polyortho-carbonates, polyphosphazenes, polyhydroxybutyrates, polyhydroxyvalerates, polyalkylene oxalates, polyalkylene succinates, poly(malic acid), poly(amino acids), poly(methyl vinyl ether), poly(maleic anhydride), chitin, chitosan, and copolymers, terpolymers, or higher poly-monomer polymers thereof or combinations or mixtures thereof, such that the initial implantation of the filler device comprises a sleeve and filler material, but over time, the sleeve is resorbed and only the filler material is left behind to augment the tissue.

In one embodiment, the sleeve comprises an outer layer of ePTFE of about 40 to 100μ pore size and about 0.001 to 0.010 inches in thickness to encourage fibrous tissue ingrowth, and an inner sleeve of polyethylene or similar material of about 0.001 to 0.010 inches in thickness to add flexibility to the sleeve and to more completely contain the filler material. Such double layer structure is particularly suited where the ePTFE is permeable or semipermeable to the filler material, such as when the filler material is, or contains a component of, water.

The sleeve may contain, or be contained by, a skeletal structure such as struts of a metal including alloys such as nitinol, stainless steel, gold, or platinum, a polymer such as PLA or PLG, or any material of sufficient durometer or structural integrity to provide support of the sleeve or to provide for a three-dimensional shape. Struts can extend in an axial direction, a circumferential direction or both depending upon the desired clinical performance. Additionally, the struts may have anchor elements or hooks which extend through the sleeve, adapted to stabilize the tissue-filling device within the tissue.

In one embodiment, the sleeve itself is highly flexible. Therefore, the material should be thin, such as within the range of from about 0.001 inches to about 0.010 inches. The sleeve may be manufactured to be of fixed length and shape, with a plurality of lengths and shapes provided in a kit, depending on the need to fill a specific region of tissue in a particular patient, or the sleeve may be cut to size at the clinical site as a part of the implantation procedure. For a given region to be filled, more than one tissue filling device may be placed to achieve a given desired contour. In one embodiment, a plurality of sleeves are provided that are bound together to create a bundle.

The tissue-filling device may be provided in a kit which includes one or more sleeves, and one or more filler materials. Or the sleeve may be supplied separately in a kit, and another kit includes one or more filler materials. Or the kit may consist solely of one or more sleeves, and the surgeon provides the filler material from an alternate source.

The sleeve may have a constant inflated diameter, generally 1-10 mm, or it may have an inflated diameter that varies along its length depending on the desired contour of the augmented tissue. For glabellar folds, the inflated diameter is, preferably, 0.5 to 2 mm. For lips, the inflated diameter is, preferably, 1.5 to 5 mm. For the upper lip, the inflated diameter preferably varies along its length adapted to form the “m” shape of the upper lip. For the lower lip, the sleeve generally tapers at the proximal and distal end, with a larger diameter of 2 to 8 mm at the central portion. In addition, for the lower lip, the profile of the sleeve will be generally a flattened “u” shape adapted to follow the profile of the lower lip. For nasolabial folds, the inflated diameter is, preferably, 2 to 6 mm, with tapering at the proximal and distal ends. In one embodiment, the sleeve comprises a series of segments such that the internal diameter of each segment is greater than the internal diameter of that portion of the lumen between segments. Further, the sleeve may have internal segmentation embodied by a series of valves or baffles. In the case of a segmented sleeve, each segment may be filled with a different volume of filler material in order to create a profile customized along the axial length of the implant to suit the specific clinical need. The sleeve may have supporting struts, such as a skeleton made from filaments, where said filaments may be composed of any biocompatible material adapted to provide structure.

A valve, or a plurality of valves, can be affixed to one or both ends of the sleeve, or along any portion of the wall of the sleeve, in order to prevent filler material from escaping into the surrounding tissue. The required integrity of the valve is dependent on the type and viscosity of the filler material. For example, if the filler material gels in place, or the filler is composed of beads of sufficient size, then the valve may not need to close tightly. In one embodiment, the valve is one or more elastomeric bands that encircles the proximal end of the sheath. In another, the valve is one or more elastomeric bands placed, during construction of the tissue filling device, 1 to 4 mm, distal from the proximal end of the sheath, and then when the sheath is turned inside out during its construction, the valve is placed on the interior portion of the sleeve, enhancing the ability of the valve to remain closed as the sleeve is filled with filler material. In another, the valve is a band of nitinol adapted to form a spring closure at the proximal end of the sheath. Other valves known in the art include, for example, U.S. Pat. Nos. 5,779,672 to Dormandy or 6,102,891 to van Erp. In addition to valve placement at the proximal end of the sheath, valves may be deployed at a plurality of locations within the sheath to form segments, which then allows individual segments to be filled with different amounts of filler material.

The filler material can be any of a number of biocompatible substances and may be of various physical states or combinations thereof, such as a non-viscous liquid, a viscous liquid, a gel, a powder, beads, flakes, continuous or discontinuous fibers, coils, fiber balls or mixtures thereof. The filler material may be transformable from a first state to permit introduction into the sheath, to a second state once inside the sheath. Combinations, such as a fiber carried within a liquid or gel are well within the contemplated scope. For example, the filler can comprise a substantially linear filament which itself can be made of a variety of materials such as nitinol, various biocompatible polymers well known to those skilled in the art, ePTFE, Proline or any biocompatible material with adequate strength to alter the contour of the tissue in which it is injected. The filler material may comprises any of a number of materials commercially available and sold as tissue fillers, such as Zyplast™, available from Inamed Aesthetics; Restylane™, available from Q-Med and Genzyme, Inc.; Hylaform™, available from Inamed Aesthetics; Artecoll™ available from Artes, Inc.; Radiance™ available from Bioform, Inc.; or Sculptura™ PLA filler available from Aventis, Inc.

Other embodiments of the filler material include a flexible random or regular coil; knit fibers; woven fabric; a series of filaments wound around each other, a compressible or non-compressible sponge material, a closed or open cell foam, or any others depending on the specific need as is well known to those skilled in the art. The filler material could be a set of objects connected with a outer membrane or an axial filament, or could be a series of discrete objects. If it is desired that the tissue-filling device be visible by x-ray or fluoroscopic imaging, then radio-opaque coatings such as triazoate, barium salts or tantalum can be included in the filler material. If ultrasonic visualization is required, small trapped air bubbles or other echocontrast material can be included in the filler material. The filler material may contain a colored dye in order to render the tissue filling device less visible from outside the tissue.

One class of fillers comprises a mix of solid particles and a carrier. One solid particle comprises micronized particles of ePTFE. Other materials that are suitable for use in the present invention include, but are not limited to, PDS II (polydioxanone, a monofilament), Nurolon (a long chain aliphatic polymer Nylon 6 or Nylon 6, 6) Ethilon (a long chain aliphatic polymer Nylon 6 and Nylon 6, 6), Prolene (Polypropylene, isotactic crystalline stereoisomer of polypropylene, a synthetic linear polyolefin), Vicryl (copolymer made from 90% glycolide and 10% L-lactide), silk, Monocryl (poly-E-caprolactone), polylactide, polyglycolide, poly lactide-co-glycolide, Medpor (biocompatible (micronized) polyethylene), BIOGLASS (bioactive glass particulate), or polyhydroxyvalerate.

Carriers that may be suitable for use in the present invention either alone, as a filler, or in combination with particles include, but are not limited to, polyvinylpyrrolidone (PVP), silicone oil, vegetable oil, saline, gelatin, collagen, autologous fat, hyaluronic acid, autologous plasma, CO₂ or other gas, and other physiological carriers.

Another class of fillers includes liquids, gas or gels without discrete solid particles. For example, PVP may be used alone or in combination with other agents. PVP is a water-soluble polyamide that possesses unusual complexing and colloidal properties and is physiologically inert. PVP is commercially available as a biocompatible gel that is freely transported through the body and is excreted unchanged by the kidneys. This gel has trade names such as Au24k and Plasdone C-15 and Plasdone C-30, and comprises macromolecules from the plasdone family, having the empirical formula (CHCH₂)₂N(CH₂)₃—CO. Polymers of this family have been used as binders, extenders, and vehicles for a variety of medications for nearly fifty years, and would be expected to be well tolerated and quickly removed from the body in the event of a valve failure, if the sleeve were to rupture or leak, or if material were mistakenly injected into the tissue, rather than into the sleeve, during the implantation procedure.

PVP is available commercially in many molecular weight ranges and is polymerized to have an average molecular weight in a particular solution. For example, PVP is available in solutions of an average molecular weight of 10,000 daltons, 40,000 daltons and 360,000 daltons. Preferably, the PVP is less than about 60,000 daltons to allow for easier renal excretion. PVP is also defined by its viscosity measurement, or K value. K values range from approximately less than 12 to 100. PVP compositions which may be desirable with the present invention are within a range of K values of from about 12 to 50. PVP is commercially available from International Specialty Products, Inc., GAF Chemical Corp., Wayne, N.J., USA, and from BASF Aktiengesellschaft, Germany. In use, the gel polymer may be diluted with deionized water or saline to produce the desired viscosity, is sterilized, and placed in cartridges for injection. Alternatively, the dehydrated polymer particles may be placed within the sleeve prior to its being placed in the tissue to be augmented, and sterile saline added after the sleeve has been placed, resulting in gel formation within the sleeve, and thence expansion of the tissue. Alternatively, the dehydrated polymer particles may be supplied in a sterile container and reconstituted with saline or water just prior to filling the sleeve.

Once the filler material is inside the sleeve, its material state or chemical structure may be altered via a number of mechanisms, such as the addition of a second material acting as a catalyst, heat or cold, change in pH, ultrasound or light, or the state change may happen spontaneously over a period of time. If the material changes its state over time, that time would ideally be in the range of 10 to 30 minutes from injection so that the clinician can mold the shape by manual palpation to a desired configuration before the filler transforms to retain its molded configuration. Alternatively, the state change would take place over 24 to 48 hours so the patient can sculpt his or her own filler configuration. In one embodiment, the filler material is a biocompatible polymer which fills the sleeve in a relatively flowable state, is molded from the skin surface by the operator to the desired shape, then light of the appropriate wave length (e.g., UV) is directed at the skin in order to convert the liquid to a non-flowable gel, which gel retains the desired suppleness. In one embodiment, the gel comprises a backbone of PEG and/or PVA, with PLA and/or PLG side groups attached to allow for biodegradability of any gel which fails to fill the sleeve or leaks out, and methylacrylates subunits attached to the backbone to induce photopolymerization with light of wavelength about 400-500 nm.

The filler material may be capable of reversing its state change, via any of the mechanisms describe above, to allow for subsequent removal of the filler material by aspiration via a channel placed in the sleeve from outside the tissue. In one embodiment, the channel is a needle which contains or is surrounded by an ultrasound crystal such that when the needle is inserted into the sleeve and energy is supplied to the ultrasound crystal, causing it to vibrate in the range of 100 khz to 1 megahertz, the gelled filler material is broken down into a flowable material allowing for aspiration through the needle.

In another embodiment, the filler material comprises a purified protein such as available from Gel-Del Technologies and described in U.S. Pat. No. 6,342,250 and U.S. Patent Application Nos. 20030007991, 20020106410, and 20020028243, which turns into a gel at body temperature and can be changed back into a flowable liquid by application of cold.

In another implementation of the invention, the tissue filling device comprises a sheath and a volume of internal foam. In this embodiment, a valve may not be required, since the foam structure itself acts to prevent filler from escaping from the sleeve. The foam may be a structure having an open or closed cell configuration. In one embodiment, the foam is a closed cell elastomer that is highly compliant, and the sheath is one of the materials noted above. The foam may be biocompatible polyurethane. The sheath may be ePTFE which is bonded to the outside of the foam. In use, the tissue filling device is placed in the tissue either directly or via the pull through sewing method previously described. Once in place, the tissue filling device is injected from a site or sites externally to the tissue to be filled, such as from the surface of the skin, with a fluid, such as water, saline, silicone, a hydrogel, or any of the filler materials described above including combinations of solid or gel particles or filaments within the fluid carrier. Preferably, a small hollow structure is used to inject the filler material, such as a 25-32 gauge hypo-tube or needle. This results in local enlargement of the tissue filling device as the closed cell foam is filled in the region in which the filler is injected. Additional sites along the tissue filling device are injected in order to customize the shape of the augmentation. If too much filler has been injected in a region, filler can be removed by re-entering the region that needs to be shrunk, and then withdrawing filler. The entry of the hypo-tube or needle into the region that needs to be shrunk can be via the same route through which the region was filled, or another pathway may be taken, such as through the skin generally perpendicularly to the axis of filling. Thus, in one embodiment, a device according to any of the embodiments described herein is selectively inflated or deflated to achieve a desired shape or contour. Alternatively, additional filler material can be added during the procedure, or at any later time as desired.

Thus, in one embodiment, filler material is added into a device (according to any of the embodiments described herein) during the implantation procedure and, optionally at least once subsequent to implantation. In another embodiment, the device is adapted to be at least partially inflated two or more times after insertion into the tissue, thereby providing a chronically adjustable device. In one embodiment, the device is implanted and inflated (e.g., filled) in one procedure or on the same day, and adapted to be further inflated (e.g., filled) on another day. These embodiments are particularly advantageous because they offer the recipient the ability to fine tune the contour and appearance of the augmentation.

The foam body is thus constructed of a cellular foam matrix having a multiplicity of cells which divide the interior volume of the implant into compartments numbering from 100 to 1,000,000 depending on the filler material chosen and the desired feel of the filled tissue. The cellular foam material may be a thermoset or thermoplastic polymer. Preferably, the cellular foam material has elastomeric qualities but may be of a non-elastomeric polymer foam. The shape of the foam body influences the basic range of shapes of the implant and for many wrinkle applications will be an elongate body having in an uninflated configuration a length of at least about 5 times and often at least about 20 times its average un-inflated cross section. The particular material or materials chosen for constructing the foam body will depend, at least in part, on the density or hardness of the tissue to be simulated.

In certain implementations, the foam body may have an “open-cell” structure, the cells being interconnected with one another by passages that permit intercellular communication of the fluid filler. The passages interconnecting the cells 20 allow the flow of fluid filler from cell to cell, which may create a hydraulic cushioning effect upon localized deformation of the implant by external pressure. The hydraulic cushioning effect created by intercellular fluid communication may help to impart realistic shape and tissue-like consistency to the implant. The viscosity of the filler at body temperature is preferably related to the passage size to inhibit excessive free flow between cells in the absence of external pressure.

The foam body may have a uniform cellular density throughout, or may have a cellular density that varies throughout one or more regions, i.e., a cellular density gradient. In the case of an embodiment that includes one or more regions 30, 32 having a cellular density gradient, the regions 30, 32 will have different average cellular densities. The average cellular density of a region can be selected to cooperate with the viscosity of the filler to influence the response of the implant to external pressure.

In another embodiment, the open cell structure may be placed within a courser closed cell structure, such that the open cell foam is compartmentalized into regions such that filler remains in a given region, and each region may be filled separately in order to vary the contour of the filled region. In one embodiment, the device according to any of the embodiments described herein, is compartmentalized and adapted to be filled separately in order to vary the contour of the filled region. In some embodiments, certain compartments are left unfilled or partially filled, and may be filled at a later date to achieve or alter a particular shape or contour.

The sleeve for the foam filled embodiment may comprise any of the materials identified previously, as well as linear aliphatic polyether urethane; linear aliphatic polyester urethane; cyclic aliphatic polyether urethane; cyclic aliphatic polyester urethane; aromatic polyether urethane; aromatic polyester urethane; polybutylene; polypropylene; crosslinked olefinic elastomers; styrene-ethylene/butylene-styrene block copolymer; or any other biocompatible material which is substantially radiolucent under standard mammographic or other imaging protocols and intensities. The fluid filler may comprise a biocompatible triglyceride, serum, saline solution, or another biocompatible material which is substantially radiolucent under standard mammographic protocols and intensities.

The foam body may also be made of a material which is substantially radiolucent under standard mammographic or other imaging protocols and intensities. The foam body may be constructed of styrene-ethylene-butylene-styrene copolymer; polyethylene; polyurethane; and polytetrafluoro-ethylene; or another biocompatible material which is substantially radiolucent under standard mammographic or other imaging protocols and intensities.

Coatings can be applied to all or a portion of any of the sleeves disclosed herein, either on the outside or the inside thereof. Methods of applying coatings to biocompatible substances are well known in the art. See, for example, U.S. Pat. Nos. 6,660,301 to Vogel, 6,368,658, and 6,042,875. The formation of and coating with hydrogels is disclosed in U.S. Pat. No. 6,652,883 to Goupil. Coatings that make the sheath sticky such as fibronectin or vitronectin or laminin can be used if desired to inhibit movement of the sheath relative to the tissue. If it is desired that the sheath be visible by x-ray or fluoroscopic imaging, then radio-opaque coatings such as triazoate, barium salts or tantalum can be used on the sheath.

Coatings can also be applied with a biologically active or therapeutic effect, as needed in the clinical application. For example, growth factors such as fibroblast growth factor, anti-inflammatory agents such as corticosteroids to reduce the amount of fibrosis, antibiotics to reduce the risk of infection on the implant, and anesthetics such as lidocaine, procaine or marcaine to decrease pain. In order to modulate fibroblast proliferation, TNP-470, a potent angiogenic inhibitor, can serve as a coating or a co-injectate. Alternatively, it may be desirable for the sheath to be coated with a tissue adhesive, such as Dermabond™, available from Ethicon/Johnson and Johnson, Inc.; or Focalseal™, available from Focal, Inc. to decrease the motion of the tissue implant device relative to the tissue. This is important since relative motion can prevent proper healing and anchoring of the device to the tissue which could eventuate in erosion. In one embodiment, the sheath is constructed of expanded polytetrafluoroethylene coated with fibrin glue containing fibroblast growth factor 1 (FGF1) and heparin.

Generally, the means for filling the sheath is provided by one or more substantially tubular structures adapted to be placed within the sheath during filling, and removable after the sheath has been filled to the desired volume. In one embodiment, the filler tube can be replaced in the sheath after its removal. The filler tube can comprise a variety of tubular structures, depending on the need, including a needle, a compliant or non-compliant plastic tube, or a metal hypotube comprised of stainless steel, nitinol, or any of a variety of materials as appropriate in view of the structure of the implant and desired filling protocol. The tube may have a variety of cross sectional profiles including round, oval, and flattened, depending on the clinical need and the shape of the sleeve to be filled.

In one embodiment, the tissue filling device is constructed and used as follows. The sheath has a proximal end and a distal end. A guide rail, which has a distal end and a proximal end, is adapted so that its distal end extends beyond the distal end of the sheath, then extends through and within the sheath from distal end to proximal end, and then emerges from the proximal end of the sheath such that the proximal end of the guide rail is proximal to the proximal end of the sheath. The guide rail is of small diameter, preferably 0.1-1.0 mm, and can comprise any appropriate filamentous material such as absorbable or non-absorbable suture, a metal such as stainless steel or nitinol, or any material or combination of materials adapted to allow a filler tube to slide over the guide rail and into the interior of the sheath. The guide rail may be coated with a material such as a hydrogel, silicone, ePTFE or PTFE to increase its lubricity.

A sew-through method of implanting the tissue filling device is as follows. A sewing needle is attached to the distal end of the guide rail using any of a number of methods as are well known in the art. The sewing needle can be straight or curved, and of small diameter, preferably 0.1-1.0 mm. Where the guide rail engages the distal end of the sleeve, the sleeve is substantially bonded to the guide rail such that filler material cannot escape from the distal end of the sleeve. The guide rail then remains unattached to the sleeve. The filler tube and attached syringe is adapted to ride over the guide rail in order for the filler tube to be placed in the sleeve, and removed therefrom after the sleeve has been filled.

In use, the surgeon measures the length of the path he wishes to fill and picks the sleeve assembly of the appropriate length from a kit of such sleeves. The sewing needle is placed by the surgeon into the skin along the path that he wishes to augment, stopping before the distal end of the sleeve emerges from the skin, and taking care that the proximal end of the sleeve is within the tissue. If it is not, the sleeve may be pulled all the way through the tissue from the distal end, thus removing it completely from the tissue. In this case, the surgeon may chose a sleeve of a different length, or may chose to enter the tissue with the sewing needle at a more proximal location, so that the entire sleeve ultimately lies within the tissue. The surgeon may put manual traction on the tissue in order to guide the needle along the desired path. The filler tube is advanced along the guide rail into the interior of the sleeve until the distal end of the filler tube is located at or near the distal end of the sleeve. A syringe with filler material is slid over the guide rail and attached to the proximal end of the filler tube. The surgeon then ejects filler material into the filler tube and thence into the sleeve. He can withdraw the filler tube along the length of the sleeve until an adequate tissue augmentation profile is achieved. The filler tube is then removed from the sleeve along the guide rail, allowing the valve at the proximal end of the sleeve to close. If more augmentation is desired, the filler tube may be again passed over the guide rail, through the valve and into the sleeve, where more filler material may be deposited. When the desired amount of filler material is within the sleeve, the filler tube is removed and the guide rail is cut flush with the skin at the proximal and distal ends of the sleeve. That portion of the guide rail within the sleeve remains there after the distal and proximal ends are cut.

In an alternative embodiment, one or more stay sutures may also be attached to the proximal end of the sleeve. In use, the stay suture extends from the proximal end of the sleeve and out to the external aspect of the tissue. The surgeon may then grasp these stay sutures to provide counterforce as the filler tube is advanced. In addition, the surgeon may grasp the stay sutures and the distal suture, or distal stay sutures if such are provided, in order to move the tissue filling device back and forth within the tissue to achieve optimal positioning. When the desired amount of filler material is within the sleeve, the guide rail is cut flush with the skin at the proximal and distal ends of the sleeve, and the stay sutures are similarly cut close to the skin at the proximal end. The stay and guide sutures are ideally of bioresorbable material as are well known in the art.

In one embodiment, the grasping means for positioning the tissue augmentation device comprises a suture, as described above. Other types of grasping means can also be used in accordance with several embodiments of the invention. In another embodiment, the grasping means comprises one or more tabs or flatted areas. In one embodiment, a portion of at least one membrane is flattened to provide the practitioner with an uninflatable area for grasping. One advantage of such an embodiment is that it may reduce the risk of damage (such as a puncture) to the inflatable portion of the augmentation device by minimizing direct contact with the inflatable portion. In one embodiment, the flattened portion or tab comprises one or more layers that are sealed using glue or another adhesive. In one embodiment, the flattened portion, or tab, is made of the same material as at least one of the membranes of the augmentation device. In another embodiment, the flattened portion, or tab, is made of a different material than a membrane of the augmentation device. The flattened portion, or tab, can be made of any shape suitable for grasping by a practitioner. In some embodiments, the tissue augmentation devices comprises a single tab. In other embodiments, the tissue augmentation devices comprises two tabs. In yet other embodiments, more than two tabs are provides. A tab may be located in any location that facilitates grasping by a practitioner. In a preferred embodiment, the tab is located at the proximal and/or distal end of the augmentation device.

In an alternative embodiment and method of use, the tissue filling device is implanted in the tissue to be augmented by means of an outer needle or cannula. The needle has a proximal end and a distal end, and a lumen extending from one end to the other. In one embodiment, the needle is 14-20 gauge. Thus, in one embodiment, the device to be implanted according to any of the embodiments described herein (e.g., the device in its first configuration or uninflated state) is sized to fit through a 14-20 gauge needle or other tubular access channel. A 14-20 gauge tubular access channel translates into a tubular access channel having an outer diameter of about 0.083 inches and an inner diameter of about 0.063 inches (14 gauge) to a tubular access channel having an outer diameter of about 0.0355 inches and an inner diameter of about 0.024 inches (20 gauge). Thus, the device to be implanted, in some embodiments, has a pre-implantation diameter in the range of about 0.024 inches (about 0.61 mm) to about 0.063 inches (about 1.6 mm). In one embodiment, the device pre-implantation or pre-inflation has diameter less than about 1.6 mm. In alternative embodiments, the device pre-implantation or pre-inflation has diameter greater than about 1.6 mm. These latter embodiments need not be delivered through a 14-20 gauge access channel.

In one embodiment, a sleeve assembly comprises the collapsed sleeve, valve and filler tube as described above. Optionally, a central guide rail may be supplied. The sleeve assembly is contained within the needle lumen such that the distal end of the sleeve assembly ends proximally of the distal end of the needle lumen. The filler tube runs through the sleeve and emerges at the proximal end of the needle, and then connected to a syringe containing the filler material. If a central guide rail is provided, the filler tube is adapted to ride over said rail. The filler material can be any of those previously described. In one embodiment, stay sutures are provided attached to the proximal end of the sleeve and emerge through the proximal end of the needle. In use, the surgeon advances the needle along the path in the tissue to be augmented from a proximally located entry site. The surgeon may put manual traction on the tissue in order to guide the needle along the desired path. The filler tube is advanced within the interior of the sleeve, and along the guide rail of such is provide, until the distal end of the filler tube is located at or near the distal end of the sleeve. The needle may be advanced through the tissue and then emerge from the skin at a distally located exit site, or the needle advancement may stop within the tissue without an exit site. In either case, once the needle is in the desired position, forward tension is placed on the filler tube to keep the collapsed sleeve in position, while the needle is retracted proximally out of the tissue. The surgeon then ejects filler material into the filler tube and thence into the sleeve. He can withdraw the filler tube along the length of the sleeve until an adequate tissue augmentation profile is achieved and may re-advance the filler tube distally if required. The filler tube is then removed from the sleeve, allowing the valve at the proximal end of the sleeve to close. If more augmentation is desired, the filler tube may be again passed through the valve and into the sleeve, and over the guide rail if one is provide, where more filler material may be deposited. When the desired amount of filler material is within the sleeve, the filler tube is removed and any guide rail and any stay sutures are cut flush with the skin at the proximal and distal ends of the sleeve.

In one embodiment, the sleeve may take the shape of the upper lip in a “cupid's bow” configuration, with the valve and filler tube assembly as provide above. The sleeve of this upper lip shape is also configurable from a first, collapsed state, to an expanded state. The sleeve of this upper lip shape may be placed within the tissue either using the sew-through method or the outer needle method described above. In this embodiment, the sleeve is generally 3 to 6 cm in length, 1 to 6 mm in width and 1 to 3 mm in depth. The upper edge has a flat “M” configuration to match the upper vermillion border of the lip. The sleeve may be constructed of two sheets of any of the biocompatible materials describe above, preferably ePTFE, attached to each other, such as by an adhesive of thermal cintering, along their edges.

In another embodiment, the sleeve is adapted to be placed in the cheek to enhance the malar fossa. In this embodiment, the shape and dimensions are well known in the art, such as described for silicone implants available from McGhan Medical Corporation, a division of Inamed. In one preferred embodiment, the sleeve is approximately ovoid and constructed of two sheets of ePTFE sintered together at their outer edges, such that the sleeve, when in its inflated state, has dimensions of 4 to 6 cm in length, 3 to 4 cm in width, and 0.3 to 1.5 cm in thickness in the center of the sleeve, with the thickness tapering towards the edges.

In one embodiment, the device is compartmentalized and the compartments are adapted to be filled separately in order to vary the contour of the filled region. In some embodiments, certain compartments are left unfilled or partially filled, and may be filled at a later date to achieve or alter a particular contour. In one embodiment, the device has two or more compartments (e.g., 3, 4, 5, 5-10, 10-20, or more than 20 compartments). As described in more detail below, these compartments can be divided by one or more interior septums. These interior septums can be pierced to inject filler and are re-sealable after a fill tube has been removed. Alternatively, each compartment (which may or may not be separated from other compartments by an interior septum) can be accessed from the exterior. Thus, the exterior can be pierced to provide filler to one or more of the compartments, which then re-seals (with or without external intervention) after a fill tube has been removed. In this manner, a practitioner can selectively fill some or all of the different compartments. The compartments can be of any size or shape (e.g., square, rectangular, circular, ovoid, elongate, triangular, amorphous, etc.). In one embodiment, the compartments are substantially flat. Thus, in one embodiment, the device for implantation into the cheek (or other suitable location) has a width of less than 3 mm. In other embodiments, the thickness is in the range of about 3 to 15 mm, as described above. In yet other embodiments, the thickness is greater than 15 mm.

In another embodiment of the invention, there is provided a tissue augmentation device comprising a generally sheet like structure formed by opposing sheets or walls joined together internally to form multiple chambers in the device. The chambers are selectively fillable, completely or partially, so as to enable the device to be shaped to a desired overall contour. The walls comprise a material that is self-sealing, so that upon withdrawal of a filling means from any chamber that chamber is self-sealed to retain the filler therein. If desired, the contour of the device may even be changed after filling one or more of the chambers by extracting filler there from.

Preferably, in this embodiment, the device comprises a pair of sheets of such self-sealing material closed together around their periphery. Such closure can be achieved by any suitable means, such as by heat or chemical bonding. More preferably, the sheets are similarly bonded together in any desired pattern to form multiple chambers or compartments.

In a preferred embodiment, either each or both of the opposing walls of the sheet device may be formed from a laminate of a plurality of layers.

The sheet, which may have self-sealing properties in a preferred embodiment, are preferably made of ePTFE and/or polyurethane.

In a related embodiment there is provided a tissue augmentation device comprising a generally sheet like or substantially planar structure comprising opposing, substantially planar walls joined together by bonding or inner walls to form a plurality of chambers therein. Preferably this embodiment has the characteristics described above. More preferably, the sheet comprises a plurality of inner chambers in an amount generally more than is needed by a surgeon for a particular application. In this embodiment, the surgeon can cut between the chambers so as to produce the desired shape and number of chambers for a particular application.

The sheet or planar chambered embodiments are particularly suitable for facial reconstructive surgery and the like.

In another embodiment, the sleeve adapted to be placed in the cheek has the dimensions described above, but additionally contains a length of Nitinol wire or ribbon in its superelastic state, of approximately 0.003 to 0.030 inches in diameter, which is affixed within the edges along the circumference of the sleeve between the sheets of ePTFE, which make up the sleeve, using a thermoplastic adhesive such as FEP or polyethylene. In such an embodiment, the sleeve is assisted in expanding from its first configuration to its second configuration, and maintaining its shape in the second configuration, by the shape memory properties of the Nitinol.

In similar fashion, other embodiments of a sleeve in the size and shape adapted to be used as tissue augmentation implants in the dorsum of the nose, the chin, the region under the eyes, the breast, or any anatomic location clinically indicated may be constructed in the fashion described above either without or with the support of a Nitinol frame structure.

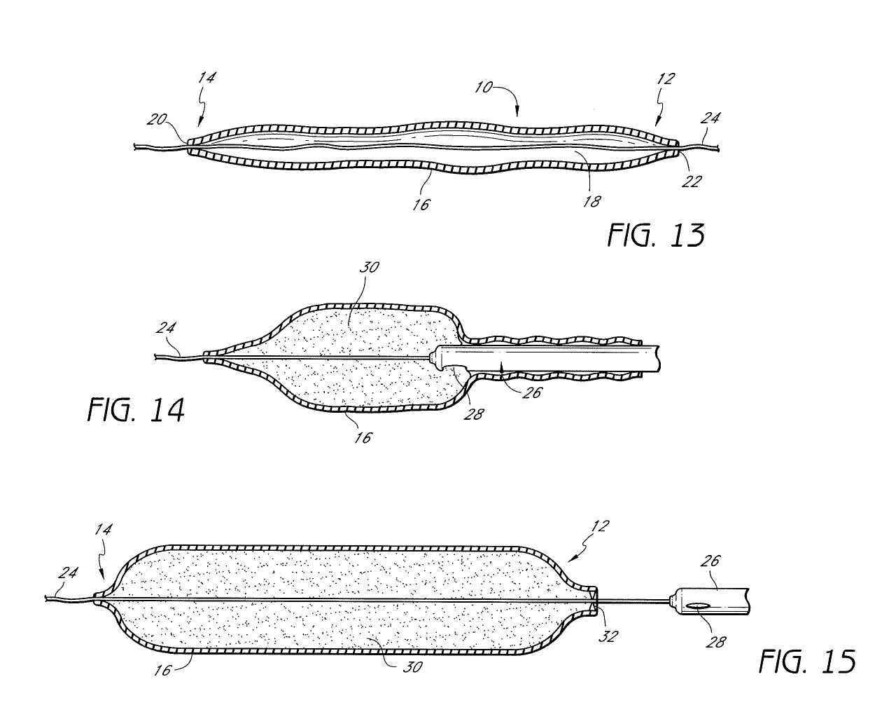

Certain specific implementations of the invention will be described with reference to FIGS. 13-24. Referring to FIG. 13, there is illustrated a schematic representation of a tissue augmentation implant in accordance with one aspect of the present invention. The implant comprises a sleeve 10, having a proximal end 12 and a distal end 14. Sleeve 10 may be either an empty sleeve with a single or plurality of macro compartments, or the outer surface of an open cell or closed cell foam as has been disclosed elsewhere herein.

The sleeve 10 comprises a body 16, which, in the present embodiment, defines a central cavity 18. The body 16 is additionally provided with a distal port 20, which is in communication with a proximal port 22 by way of a lumen extending therebetween. In the illustrated embodiment, the distal port 20 is on a distal end of the body 16 and the proximal port 22 is on the proximal end of the body 16. However, either port may be positioned along the length of the body 16 spaced apart from the respected end, depending upon desired performance and other design considerations. A plurality of ports may also be desirable.

In the illustrated embodiment, the distal port 20 and proximal port 22 serve as guidewire access ports to allow the body 16 to be slideably advanced along a guidewire 24.

The illustrated ports 20 and 22 are in communication with each other by way of the central cavity 18. However, a separate lumen may be provided through the sleeve wall or on the outside of the sleeve if it is desired to isolate the guidewire lumen from the filler media.

As has been discussed herein, the body 16 is transformable from a reduced cross sectional configuration such as for positioning at a desired treatment site, to an enlarged cross sectional configuration for providing a desired cosmetic result. In one embodiment, illustrated schematically in FIG. 14, the body 16 is transformed to the enlarged cross sectional configuration by filling the central cavity 18 with any of a variety of desired filler materials 30. A filler tube 26 is advanced along the guidewire 24 to position a fill port 28 within a desired portion of the central cavity 18. The proximal end of the filler tube 26 (not illustrated) is connected to a source of filler media, such as a hypodermic needle syringe or other container depending upon the nature of the filler media. Suitable filler materials are disclosed elsewhere herein, and the nature of the filler tube may be modified to take into account the nature of the filler as will be apparent to those of skill in the art in view of the disclosure herein.

The filler tube 26 may be advanced throughout the length of the sleeve 10 into the vicinity of the distal end 14. Filler 30 may be deployed through the fill port 28 by activation of a fill control (not illustrated) on the proximal control. The filler tube 26 may be axially proximally retracted through the sleeve 10 to introduce filler 30 at different positions along the length of the sleeve. After a sufficient amount and desired distribution of filler 30 has been introduced into the sleeve 10 to achieve the desired result, the filler tube 26 may be proximally retracted from the proximal end 12, and removed from the patient. See FIG. 15. Proximal end 12 may be provided with a valve 32 as has been described herein, to permit removal of the filler tube 26 and retention of the filler media 30 within the sleeve 10. The guidewire 24 may also thereafter be proximally withdrawn from the sleeve 10, thereby leaving the filled implant in position at the desired treatment site.

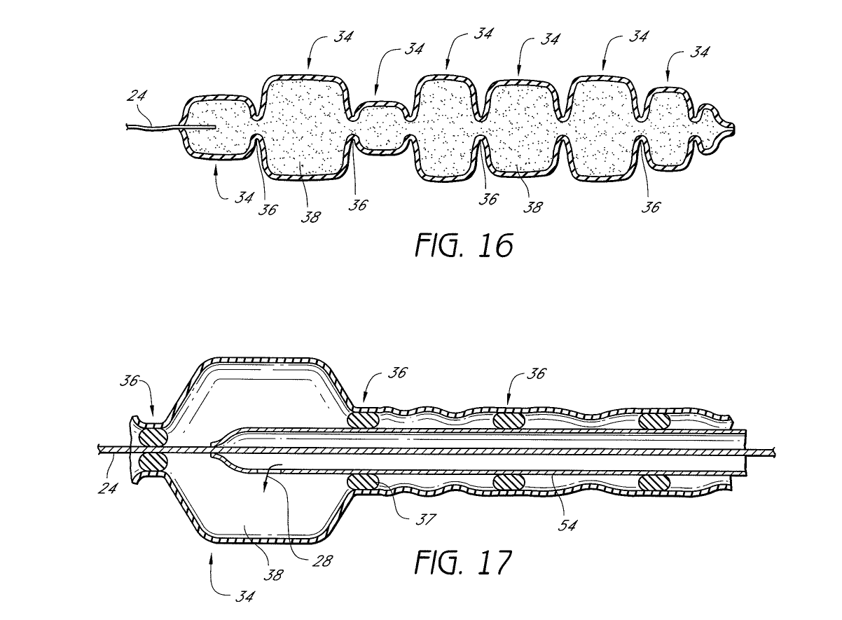

For certain applications, the sleeve 10 is preferably fillable to a non-uniform profile. This may be accomplished utilizing the embodiment of FIGS. 13-15, together with a filler which has sufficient viscosity, or structural characteristics (e.g. wire coils) that the filler will remain at a localized position within the sleeve 10. Alternatively, referring to FIG. 16, there is illustrated a segmented embodiment of the invention. The sleeve 10 is divided into a plurality of segments 34, which are separated by a plurality of neck portions 36. The fill port 28 on the fill tube 26 may be sequentially positioned within each of the segments 34, to allow each segment 34 to be inflated to a unique cross sectional dimension. In this manner, the cross sectional dimensions of the implant are customizable along the length of the implant as may be desired to achieve a desired cosmetic result.

The neck portion 36 may be formed in any of a variety of ways, such as by heat forming the sleeve 10, or by placing any of a variety of structures such as a band around the neck portion 36. Referring to FIG. 17, the segmented implant is illustrated with a filler tube 26 in place within a segment 34. Adjacent segments 34 are separated by a restriction 37 such as an annular elastic band or gasket. The restriction 37 has sufficient elasticity to permit passage of the filler tube 26, but recoils back to close of substantially close the passageway between adjacent segments 34 following removal of the filler tube 26. Thus, the restriction 37 may be configured to either restrict and control flow between adjacent segments 34, or completely block flow of filler 30 between adjacent segments 34.

The nature of the restriction 37 in neck portion 36 is configured to cooperate with the nature of the filler 30 as will be appreciated by those of skill in the art in view of the disclosure herein. For example, the restriction 37 need not provide a rigorous seal if the filler 30 comprises a plurality of coils, fibers, or particular material. However, if a less viscous or more flowable filler 30 such as saline solution is utilized, restriction 37 should be configured to provide a seal between segments 34 if it is desired to prevent flow of filler 30 between adjacent segments 34. Optimization of these parameters may be achieved through routine experimentation by those of skill in the art, taking into account the desired clinical performance of the implanted device.

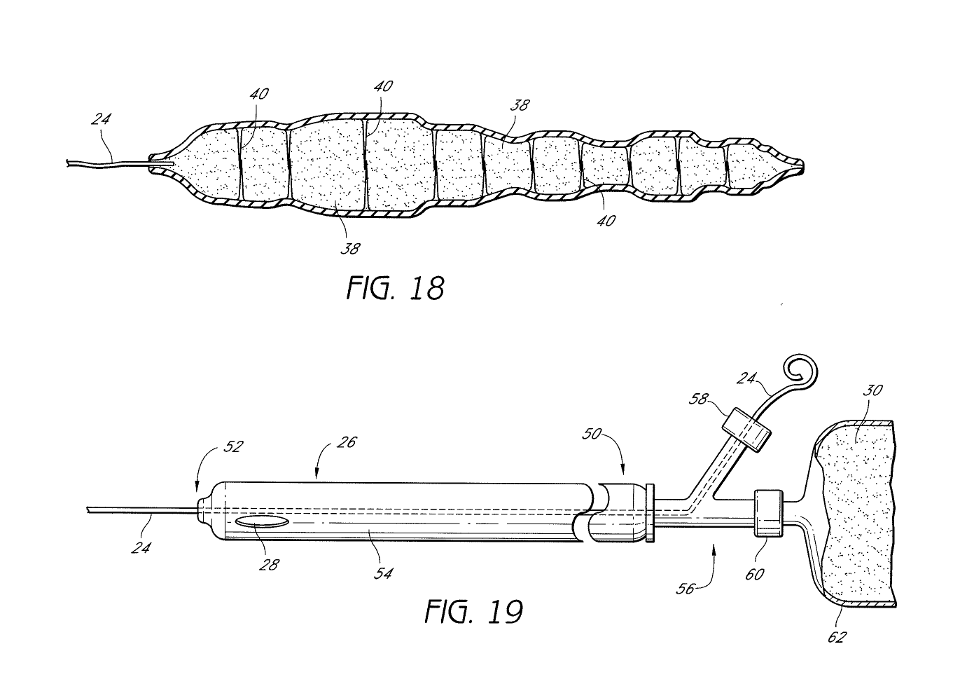

Referring to FIG. 18, a sleeve having a plurality of internal baffles 40 is disclosed. Baffles 40 function to divide the interior cavity 18 of the sleeve 10 into a plurality of chambers or compartments 38, without necessarily influencing the external profile of the implant. Similar to the restriction 37, baffles 40 permit the filler tube to be advanced and retracted to reach each compartment 38, and then to prevent or to substantially prevent the flow of filler 30 between adjacent compartments depending upon the desired clinical performance. As a further alternative, the baffles 40 or valves may be in the form of a pierceable septum, which permits passage of the fill tube 26 but which reseals either completely or substantially following removal of the filler tube 26. Alternatively, each chamber or compartment (which may or may not be separated from other compartments by an interior septum) can be accessed from the exterior. Thus, the exterior can be pierced to provide filler to one of the compartments, which then reseals (with or without external intervention) after a fill tube has been removed. In this manner, a practitioner can selectively fill some or all of the different compartments to achieve a desired profile or contour.

Referring to FIG. 19, there is illustrated one embodiment of a filler tube 26 in additional detail. Filler tube 26 comprises a proximal end 50, a distal end 52 and an elongate tubular body 54 extending therebetween. Tubular body 54 may be flexible or rigid, depending upon the desired performance. Tubular body 54 may be formed in any of a variety of ways, such as by machining from metal components (e.g. stainless steel hypotube) or by extruding any of a variety of polymeric materials well know in the catheter arts, such as PEEK, PEBAX, various densities of polyethylene, among others.

The tubular body 54 includes at least one central lumen for receiving the guidewire or guide rail 24 therethrough. The guidewire lumen is in communication with a guidewire access port 58 on the proximal manifold 56. Proximal manifold 56 is additionally provided with a filler port 60, which may be a lure connector or other quick release hub, for removable connection to a source 62 of filler 30. In one convenient embodiment, source 62 is in the form of a manually activatable syringe.

The tubular body 54 may be provided as a dual lumen structure, having either concentric or side-by-side lumens as is well known in the catheter arts. Alternatively, depending upon the nature of the filler 30, the guide rail 24 may extend through the same lumen as the filler media as well be appreciated by those of skill in the art in view of the disclosure herein.

Although the filler tube 26 is illustrated as having a single effluent port 28 for introducing filler 30 into the sleeve 10, a plurality of filler ports 28 may be provided. In addition, the filler port 28 may be the same as the distal opening through which the guide rail 24 extends. In an embodiment having multiple effluent ports 28, the multiple ports may be arrange circumferentially in a single transverse plane about the tubular body 54, or may be spaced axially apart along the length of the tubular body 54 such as for use in a procedure where it is desired to fill multiple compartments 38 simultaneously.

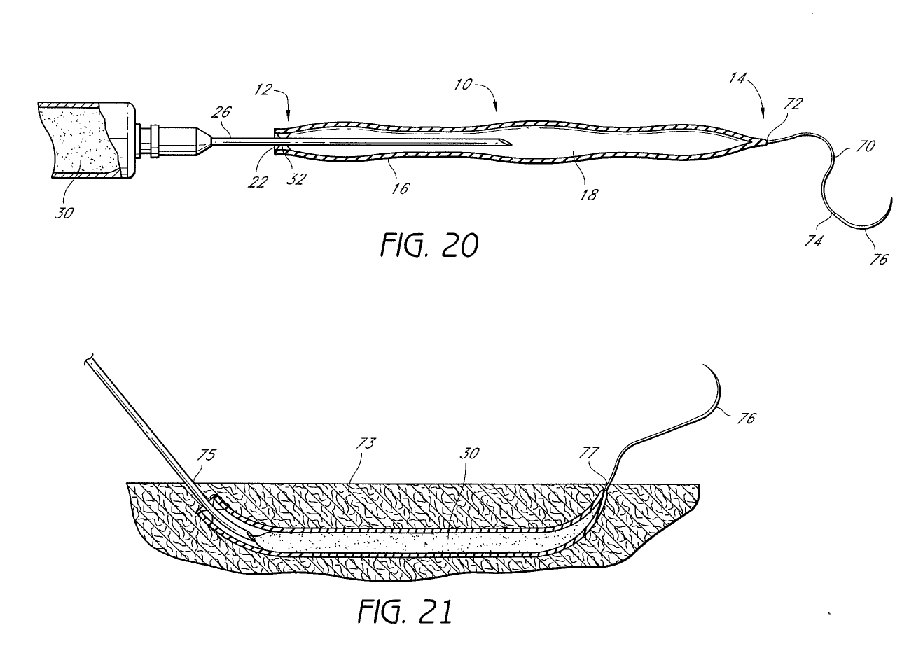

A further implementation of the invention is illustrated in FIG. 20. A schematically illustrated sleeve 10 extends from a proximal end 12 to a distal end 14. The sleeve comprises a flexible body 16 which may comprise an outer fabric sleeve or the outer surface of a segment of foam, as has been discussed elsewhere herein. In the illustrated embodiment, the body 16 defines at least one central cavity 18, having a proximal port 22. Proximal port 22 is provided with a valve 32, for sealing the central cavity 18 following introduction of filler material 30 and removal of the filler tube 26.

In the implementation of the invention illustrated in FIG. 20, the distal end 14 of the sleeve 10 is provided with a closed end. A distal suture 70, extending from a proximal end 72 to a distal end 74 is attached to the closed distal end 14 of the sleeve 10. In alternative embodiments, distal end 14 may be provided with an open access port, with or without a valve, depending upon the desired filling configuration. The suture 70 may also extend throughout the length of the sleeve 10, and proximally from the proximal end 12 of sleeve 10, depending upon the desired performance.

In the illustrated embodiment, the distal suture 70 extends from the distal end 14 of the sleeve 10, to a needle 76 attached to the distal end 74 of the suture 70. Needle 76 may comprise any of a variety of sewing needles, as will be apparent to those of skill in the art in view of the disclosure herein.

FIG. 21 schematically illustrates the use of the embodiment of FIG. 20. The needle 76 is introduced into the skin 73 at a first access point 75. The needle is advanced subcutaneously beneath an area to be treated. Needle 76 is thereafter advanced through the surface of the skin at an exit point 77. Further traction on the needle 76 and suture 70 pull the tubular sleeve 10 through the entrance point 75 and into position beneath the region of skin to be treated. Once the sleeve 10 is in the desired position, the filler material 30 is advanced from a source into the central cavity 18. Following introduction of a desired volume of filler material 30, the filler tube 26 is proximally withdrawn from the sleeve 10, and the distal suture 70 is severed at or below the skin surface.

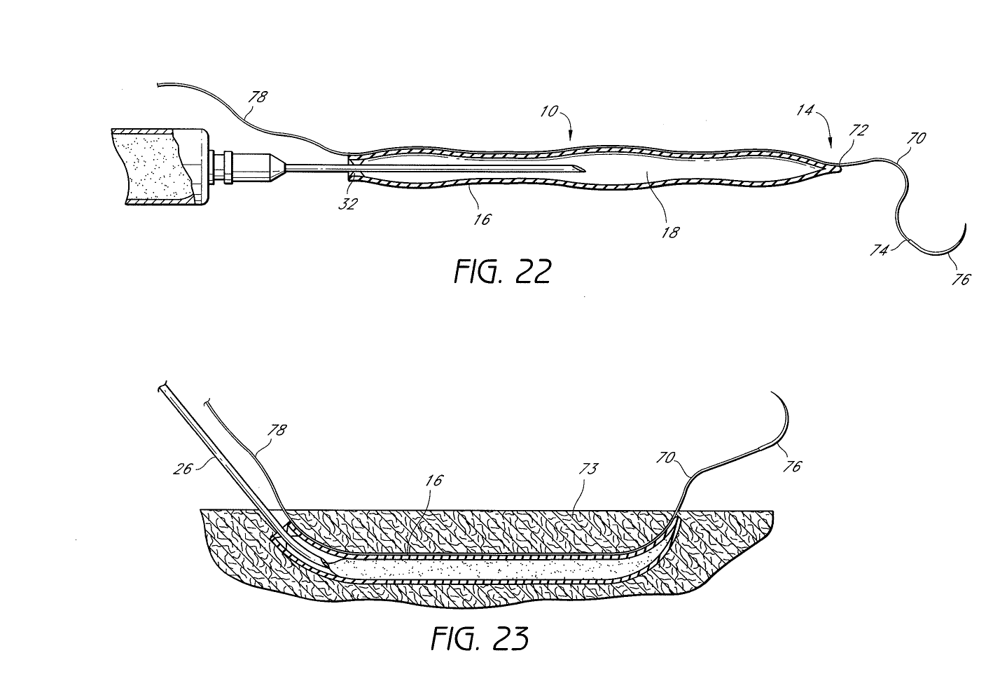

Referring to FIGS. 22 and 23, there is illustrated an embodiment like that in FIGS. 20 and 21, with the added feature of a proximal stay suture 78. Proximal stay suture 78 may be attached to the sleeve 10 in the vicinity of the valve 32, or may be a continuous suture with the distal suture 70, extending along the outside or the inside of the body 16.

In use, the proximal stay suture 78 and the distal suture 70 may be used to manipulate the sleeve 10 along its axis to optimize positioning either before, during or following introduction of filler material 30 into the central cavity 18.

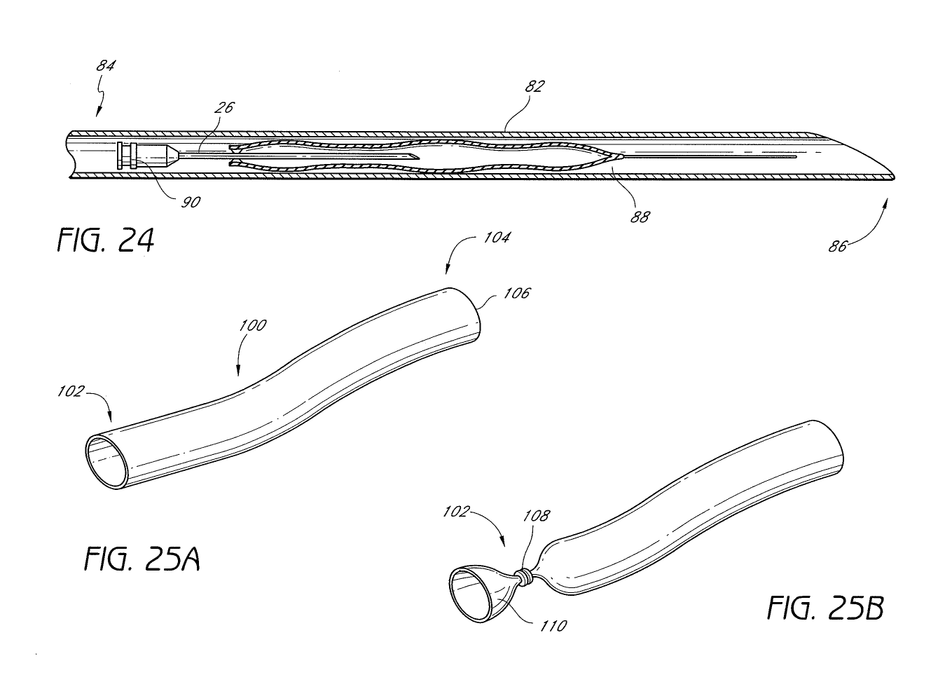

A schematic representation of the use of an external introduction needle is illustrated in FIG. 24. In the present context, the use of the term “needle” is not intended to imply any specific structural dimensions, other than as necessary to provide access for subcutaneous insertion of the implant. The actual dimensions of the introduction needle will be optimized for or governed by the configuration of the implant and filler tube as will be apparent to those of skill in the art.

Placement needle 82 comprises an elongate tubular body 83 extending between a proximal end 84 and a distal end 86. Tubular body 83 comprises an elongate central lumen 88 extending therethrough. The tubular body 83 may comprise any of a variety of forms, depending upon the intended clinical use. For example, tubular body 83 may comprise a straight, a curved, or a flexible configuration. Typically, the distal end 86 will be provided with a bevel or other sharpened tip, to facilitate advance through soft tissue. Depending upon the diameter of the tubular body 83, a separate obturator tip may be positioned within the tubular body 83 to facilitate positioning of the tubular body 83 in the desired treatment site. The obturator may thereafter be removed, and the sleeve 10 advanced into position within the tube.

In the embodiment schematically illustrated in FIG. 24, the tube 83 has a sufficient inside diameter to accommodate a proximal hub 90 on the filler tube 26. This allows the placement needle 82 to be proximally retracted over the assembly of the sleeve 10 and filler tube 26 following placement at the treatment site. Alternatively, the placement needle 82 can be configured to be withdrawn in a distal direction out of the exit point 77 (see FIG. 21). Thus, depending upon the desired clinical performance, the placement needle 82 may be proximally retracted or distally advanced off of the sleeve 10. In an alternate configuration, placement needle 82 may be in the form of a peel-away sheath, which can be removed proximally without the need for an inside diameter sufficient to accommodate the proximal hub 90. Any of a variety of configurations may be utilized for the placement needle 82, as will be apparent to those of skill in the art in view of the disclosure herein.

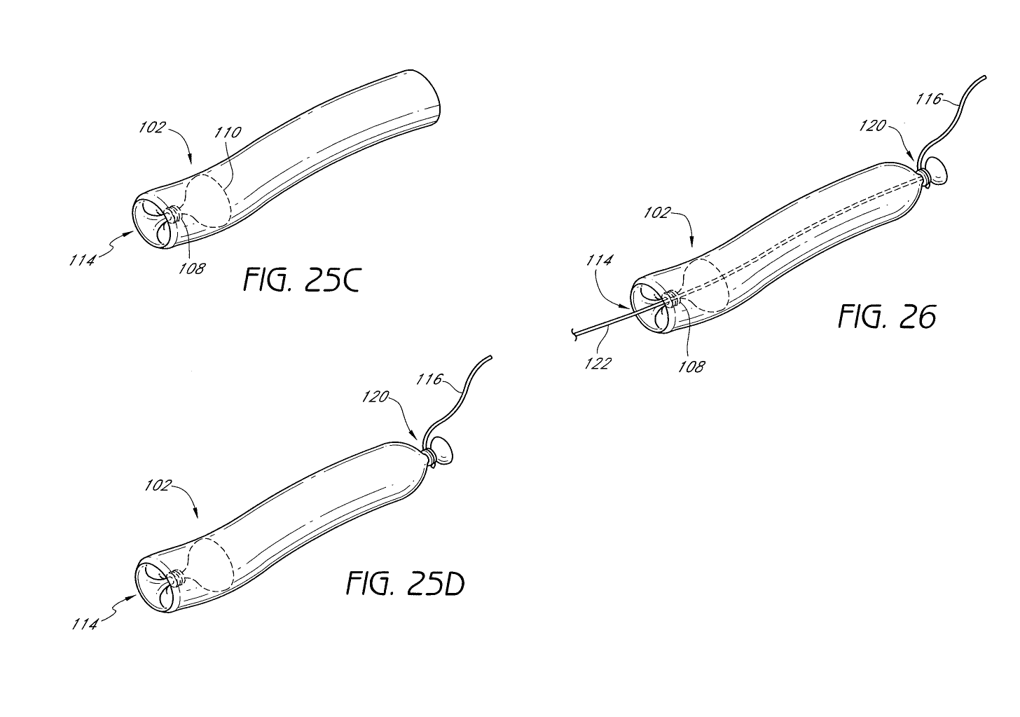

Referring to FIGS. 25A through 25D, there is illustrated a manufacturing sequence for a tissue augmentation device in accordance with the present invention. FIG. 25A illustrates a tubular sleeve 100 which extends between a proximal end 102 and a distal end 104. A central lumen 106 extends therethrough. Tubular sleeve 100 may comprise any of a variety of materials such as ePTFE and others described elsewhere herein. In general, tubular sleeve will have a sufficient length and diameter to accommodate the desired treatment site. For treatment of wrinkles in the face, tubular sleeve 100 will generally have a length within the range of from about 1 cm to about 6 cm, and a diameter within the range of from about 1 mm to about 8 mm. The wall thickness of the tubular sleeve 100 may also be varied considerably, but will often be within the range of from about 0.003 to about 0.020 inches.

Referring to FIG. 25B, there is illustrated the first step in construction of the proximal valve 114. A biasing element 108 such as an elastic band, suture, spring biased metal clip, or other clamp or biasing member is positioned around the tubular sleeve 100 to create a neck, spaced slightly apart from the proximal end 102 leaving a trailing end 110 of the tube 100. The biasing element 108 is preferably sufficiently tightly positioned around the tube 100 to provide a suitable seal taking into account the desired filler material as has been discussed.

As seen in FIG. 25C, the tubular body 100 is then turned inside out (everted) so that the trailing end 110 is positioned within the central lumen 106. The biasing element 108 is also positioned within the central lumen 106, presenting a valve opening 114 on the proximal end 102 of the tubular body 100. Valve opening 114 permits the introduction and removal of a filler tube as has been discussed.

Referring to FIG. 25D, a distal closed end 120 is formed on the tube 100. Closed end 120 may be provided in any of a variety of ways, such as by one or more loops of a suture 118 which may be tied into a knot. Alternatively, any of a variety of adhesives, thermal welding, elastomeric bands, clips or other biasing structures such as those utilized to form valve 114 may be used. In the illustrated embodiment, closed end 120 is provided by tying a suture tightly around the distal end 104 of the tube 100. A trailing end 116 of the suture is left attached to the suture knot, to provide assistance during positioning as has been discussed. The distal suture 116 may thus be provided with a sewing needle (not illustrated) for percutaneous introduction into the treatment site.

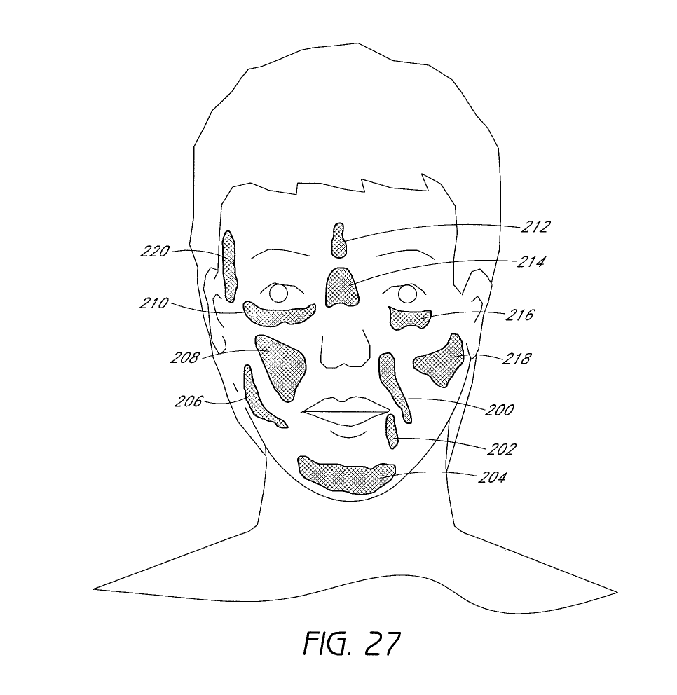

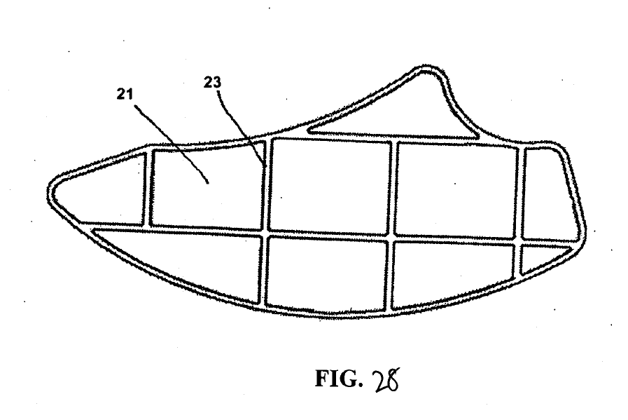

Referring to FIG. 26, there is illustrated a tissue augmentation device as in FIG. 25D, with an optional guidewire 122. Guidewire 122 extends through the valve 114, and at least as far as the distal closed end 120. Guidewire 122 may be permanently attached, at the closed distal end 120, or may be removable such as by proximal traction depending upon the desired clinical performance. In one embodiment, the guidewire 122 is secured within the suture knot 118 and not intended for removal. In this embodiment, following placement and filling of the implant, the proximal portion of guidewire 122 is severed at about the valve 114. Guidewire 122 may comprise any of a variety of filaments, such as a suture, or a metal wire such as stainless steel or Nitinol. As has been discussed, guidewire 122 may provide assistance in axial repositioning or positioning of the filler tube, which may be advanced over the guidewire 122 and into the tubular sleeve 100.

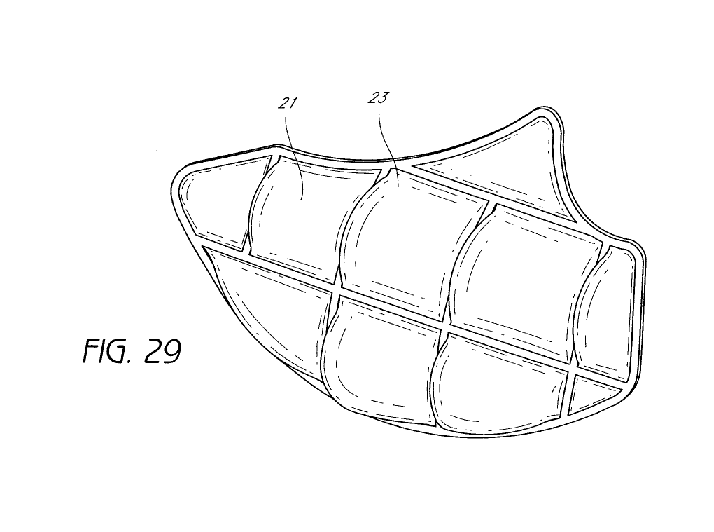

Various embodiments of implants disclosed therein, both of a generally cylindrical form and of a generally sheet-like form, may be implanted in several locations throughout the body. Some specific possible implant locations on the face are illustrated in FIG. 27 below.

Referring to FIG. 27, implants of the disclosed design are also useful in the periorbital region, such as the suborbital rim 210, including the more medial portion of the suborbital zone known as the tear trough 216. Depending on physician preference and patient anatomy, an implant for this location can be similar to either the elongate, generally linear implants such as those used for the nasolabial region 200, or can be of a more sheet-like planar nature and extend inferiorly to the region in which the malar prominence meets the cheek or medially to the region in which the malar prominence meets the nose (the nasojugal region).

Wrinkles in the glabellar region 212 can be corrected with an implant of the disclosed design as well. As described in reference to the periorbital region, the particular anatomy of this region and physician preference will determine whether a linear or planar implant is best suited, as either can be effective for the types of defects or wrinkles found in this region.

In another embodiment, the bridge of the nose 214 can be augmented with a planar-type implant of the disclosed design. Use of such an implant can be particularly effective in patients who have a flattened nasal bridge but desire a more prominent nasal bridge.

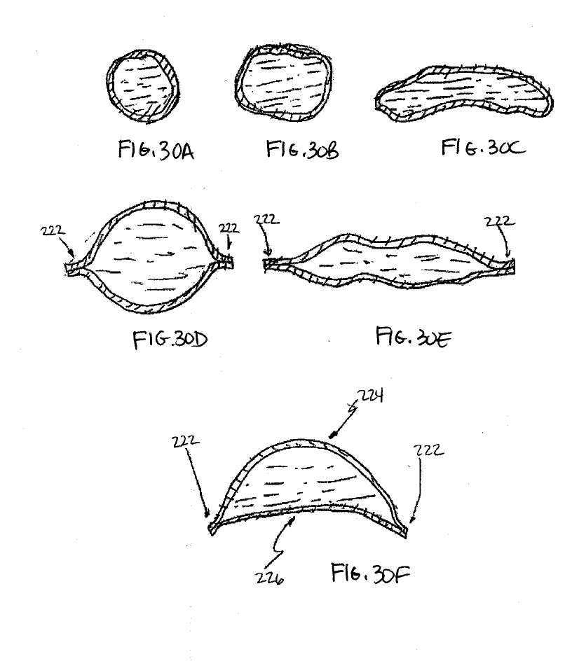

In one embodiment, augmentation of the malar and submalar regions 218 by use of an implant of the disclosed design can be very effective in reshaping a patient's face through the alteration of the underlying structure on which the overlying soft tissue is draped. Implants in this location would benefit from use of the disclosed self-sealing chronically adjustable membrane, as would all of the other implants described herein. FIG. 28 is a plan overhead view of a deflated mid-face malar implant, while FIG. 29 illustrates the same implant in inflated condition. Both figures illustrate an embodiment with a plurality of internal segments 21, which may be separated by a series of valves or baffles 23 (e.g., seams formed by thermal bonding, adhesives, etc.) described above. Furthermore, a planar implant may also be used in the temporal region 220.

FIGS. 30A-F shows cross-sections of various preferred tube-based and sheet-based implants in various configurations that can be utilized depending on the desired implant location, specific contours of the patient's face, and physician preference. FIG. 30A illustrates the configuration of a tube-based, taut-filled implant. FIG. 30B shows a tube-based, slightly flaccid implant. FIG. 30C shows a tube-based markedly flaccid-filled implant. FIG. 30D shows a sheet-based, taut-filled implant. FIG. 30E discloses a sheet-based, flaccid filled implant. FIG. 30F shows an example of a sheet-based implant with two sheets of differing compliances (or two sheets of similar compliance but dissimilar area) that may be desirable in order to make an asymmetric cross-section upon inflation. Sheet layer 224 shown is desirably constructed of, for example, a relatively low modulus elastomeric film that is highly compliant, producing a more curved shape. Sheet layer 226, in contrast, may be made of a relatively higher modulus, less compliant elastomer/polymer. The sheet-based implants may all use bonded seams 222 to connect the two sheet layers together.

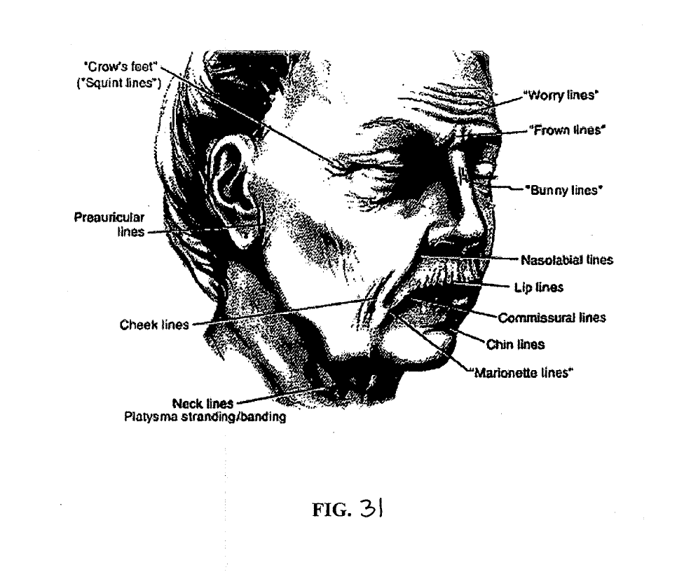

In one embodiment, an implant 200 (FIG. 27) that is of an elongate nature may have a cross-section that is either substantially round or a flattened shape (when flaccidly filled) as previously disclosed, and illustrated in FIGS. 30A-C. Alternatively, the implant may be constructed using a sheet-based method to product cross-sections such as those shown in FIGS. 30D-F. Such an implant is well suited to effacement of the nasolabial groove. A similarly shaped but shorter implant 202 can be used for the marionette lines (or “pre jowl sulcus”). In both of these cases, the implant is used to augment the soft tissue that lies beneath a line-like feature in the patient's skin, commonly referred to as a wrinkle or rhytid. Similarly, implants of this type will be effective at any of the locations shown in FIG. 31, illustrating the terminology and anatomical location of typical sites of facial wrinkles. Length, diameter, cross-sectional shape and overall shape, such as whether the elongate shape is linear or arcuate, bulbous, tear-drop shaped or otherwise curved, are ideally chosen to best suit each of these locations. Alternatively, many of the wrinkle locations on many patients may be effectively effaced through the use of an implant selected from a kit which includes a relatively small number (e.g. at least about three or four and often no more than about 5 or 10) of more generically shaped implants that are made available in incremental lengths and diameters.

In another embodiment, a deeper implant 204 can be used for the several locations in the chin (mental) region or other portions of the mandible. The implant will have a curved shape if used to augment the central mentum in order to match the natural curvature of the bone, but is less curved if used to augment the posterolateral mandible 206 also referred to as the angle of the mandible or the pogonion. Implants such as this will generally be formed using sheet-based methods and be implanted at a relatively deep tissue plane, such as just supraperiosteal or infraperiosteal (just on or below the outer surface layer of the bone). In some cases, however, physicians may, depending on aesthetics and the unique characteristics of the patient choose to place these implants in the same subdermal plane used for the wrinkle-correcting implants such as those described above.

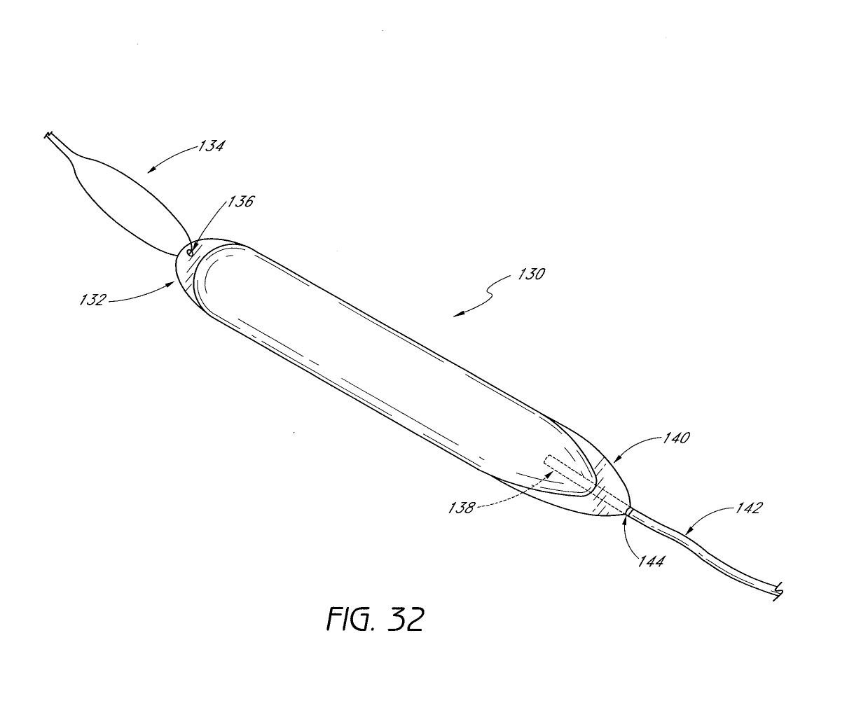

Also disclosed in the present invention is a tissue augmentation device specially tailored for nasolabial implantation. The method of implantation, as well as a specialized grasper to assist in implantation, will also be described. In this embodiment, there is a tissue augmentation device with size and shape that is preferred for placement below the nasolabial area of a patient's face in order to efface the crease-like appearance of that area. Other sizes and shapes may be preferred for other facial zones as described below. FIG. 32 shows one embodiment of an inflatable tubular implant in its deflated state as it would be seen prior to insertion into the soft-tissue (such as the nasolabial region) of a patient. The body 130 of the implant constitutes the central portion and is responsible for the majority of the volume augmentation that the inflated implant provides. A distal tab 132 is provided to allow for insertion and manipulation of the implant without directly grasping or otherwise attaching to the implant body. In this embodiment, “distal” refers to that end being farther away from the typical point of entry. In the case of the nasolabial area, the distal end of the implant will typically be positioned in the perialar zone (near the patient's nostril) and will thus also be referred to as the superior or cranial ends.

In one embodiment, also shown in FIG. 32, the distal tab 132 is provided with a through-passage such as a punched hole 136 which allows passage of a suture 134. The hole 136 may be provided with a reinforcement structure, such as a metal or polymeric ring to reduce the risk of the suture pulling through the distal end of the tab 132. Alternatively, a filament such as a wire can extend axially along a first side of the implant, loop around the distal side of the hole 136 and then extend axially along a second side of the implant. Alternative reinforcing structures may also be used.

The suture 134 may be formed into a loop as shown here or may pass through the tab and then be knotted in typical fashions. The suture 134 allows the physician, who may also be an assistant, or other operator, to apply traction to the distal end of the implant for purposes of either providing location and orientation control during and after the implantation procedure, drawing the implant into the tissue to implant it (in what is referred to as the “sew-through” method), or both. The suture 134 connects on its other (distal) end, not shown, to either a surgical needle or to a delivery system which may combine needle elements with other delivery system elements, such as dissection components for sharp dissection, blunt dissection or both.

The distal tab 132 meets the body of the implant 130 along a generally arcuate path, said path being formed during the fabrication process by controlled application of adhesive, heat-bonding or other suitable bonding means, under compression using a curved tool. The generally circular path of the edge of the bond causes the implant to inflate at its distal end into a bulbous shape. This design is advantageous in that it more closely matches the required tissue augmentation of this region of the nasolabial area: the subdermal tissue plane in which the implant is optimally placed is at its deepest in this location relative to the rest of the nasolabial region, from approximately 2 mm to 6 mm deep to the skin, and the depth and extent of the nasolabial crease (which can also be referred to as the nasolabial fold or the nasolabial sulcus) is at its greatest in this location. A distal tab 132 may have an axial length of at least about 1/16″, ⅛″, ¼″, ½″, or more, and may further enable grasping by a grasping tool, described further below.

In another embodiment, a proximal tab 140, with or without a reinforcing element may be provided as well to allow further locational and orientational control of the implant during and after the implantation procedure. The proximal tab 140 also provides a fixation zone for the valve assembly 138, which may be fixated to the inside materials of the proximal tab 140 by adhesive, heat-bonding or other suitable bonding means. A proximal tab 140 may have an axial length of at least approximately 1/16″, ⅛″, ¼″, ½″, or more to enable grasping by a grasping tool, described further below.

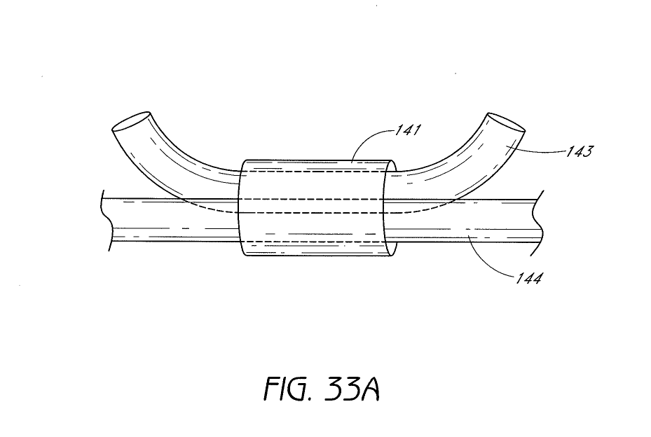

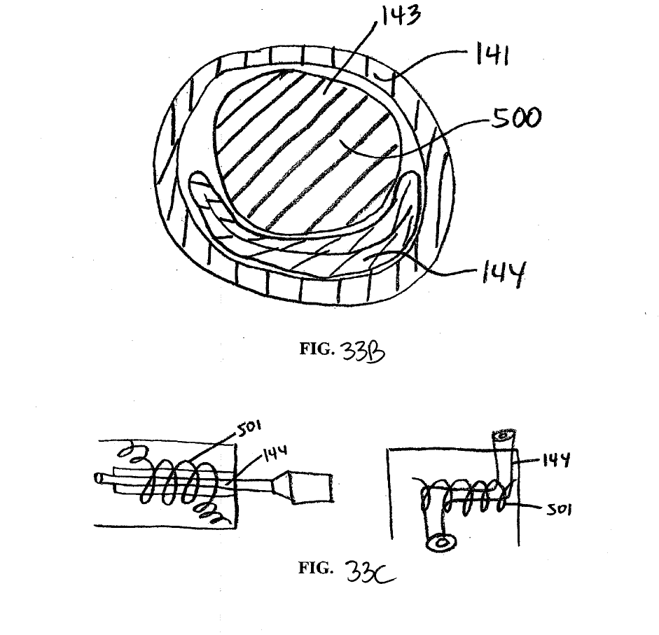

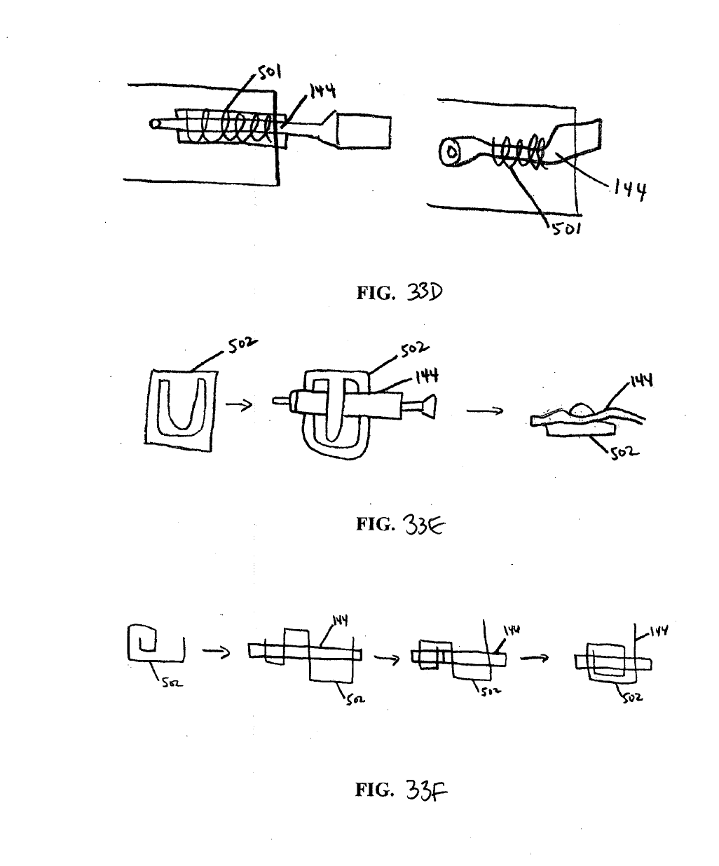

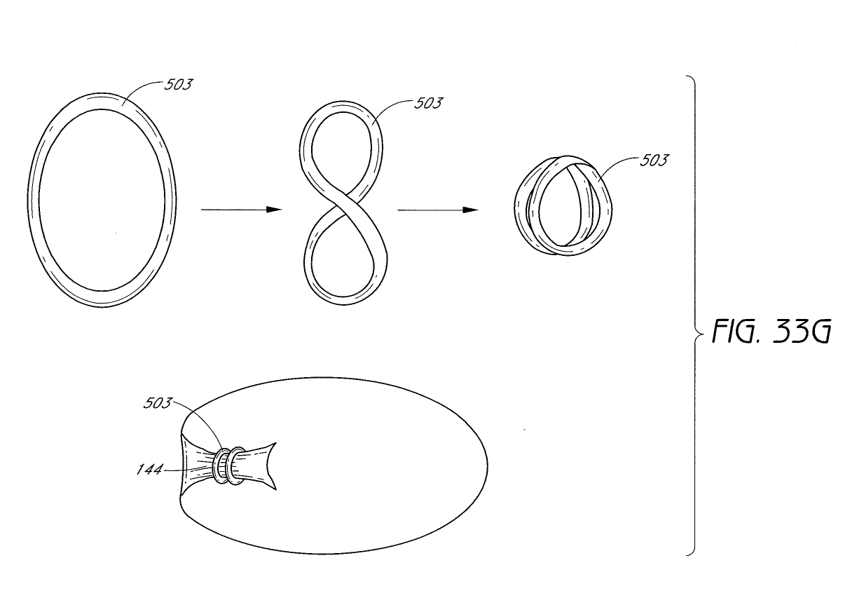

The most proximal portion of the valve assembly is the neck tube 144 into which the fill tube 142 is inserted. FIGS. 33A and 33B illustrate the components of the valve assembly. The valve assembly consists of a neck tube 144 surrounded by an elastomeric valve tube 141 or other spring-like material such as stainless steel, spring steel or superelastic NiTi. Positioned within the elastomeric tube 141 and adjacent the tube 144 is a valve plug 143. In FIG. 33B, an exaggerated cross-sectional view of the valve assembly (to show the functional relationship between the valve plug 143 and neck tube 144, a cylindrical valve plug 143 positioned within an outer sleeve of the valve tube 141, causes the neck tube 144 to collapse in a “crescent-moon” shape in response to the inward radial constriction of the valve tube 141. Without the valve plug 143, the neck tube 144 would constrict in sphincter-like fashion thus allowing leakage along the small folds inherent in that type of collapse. The valve plug 143 may contain or comprise filler material 500 such as compressible foam or an elastomeric rod holding the valve tube collapsed unless the filling cannula is in position therein.

Depending on the desired long-term performance of the valve assembly, it may be desirable to add an additional sealing component, such as a nitinol or stainless steel, clip or a seal by heat sealing or application of an adhesive after filling, to maintain the competency of the valve. Some additional sealing components are depicted in FIGS. 33C-G. FIG. 33C is a valve with a nitinol coil restraint 501, while FIG. 33D is another valve 144 with a nitinol coil restraint 501 in a different configuration, according to one embodiment of the invention. FIG. 33E is a valve 144 with a “paper clip” configuration of the nitinol coil restraint 502, while FIG. 33F is another variation of a nitinol coil restraint 502, according to another embodiment of the invention. Another alternative embodiment of an additional sealing mechanism is shown in FIG. 33G, a valve 144 with a folded O-ring 503 configuration. Any other variety of additional steps such as sealing, clamping, locking, gluing, radiofrequency welding, or ultrasound may also be utilized. Furthermore, in another embodiment, a thermal source such as laser, electricity, flame, or a heating loop may be utilized to melt the valve shut, such as a flange with a wire loop attached to the fill tube and proximally connected to a power source, such as a battery.

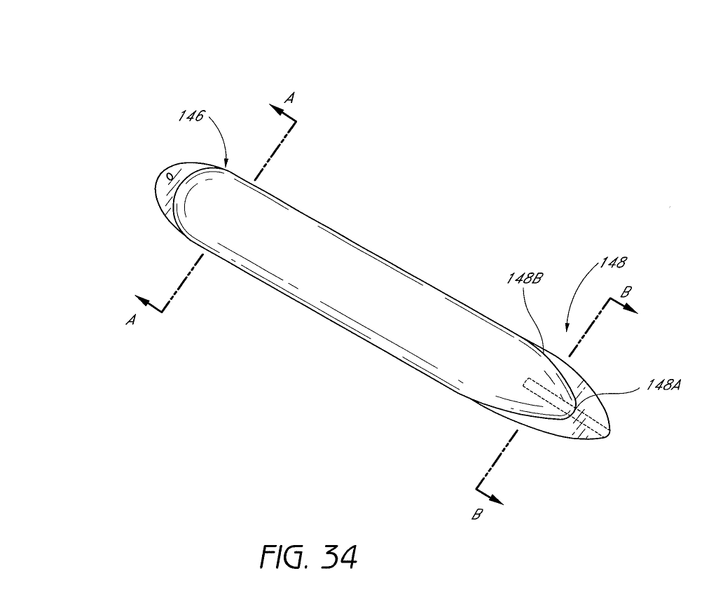

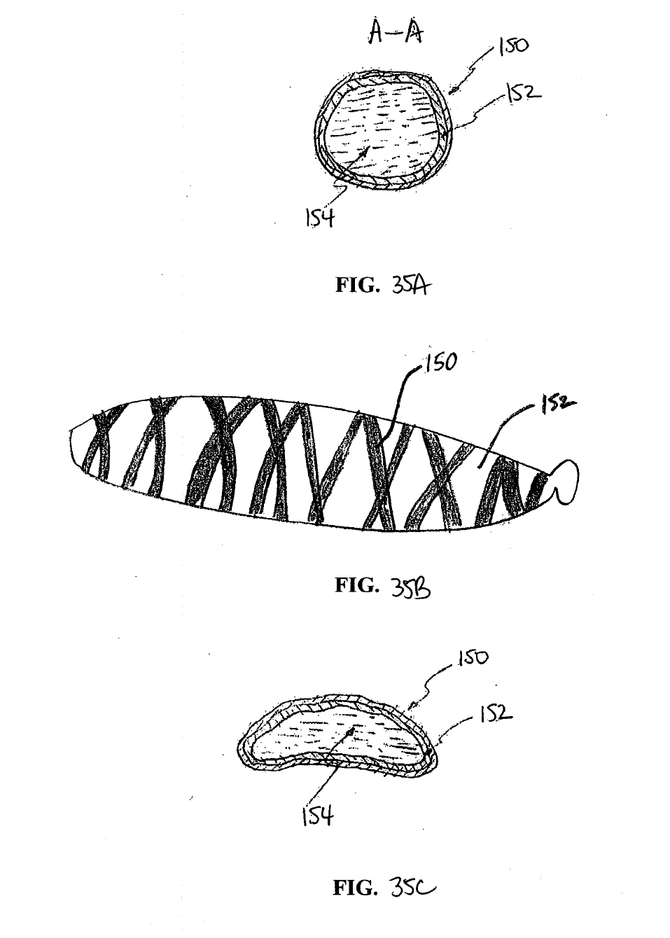

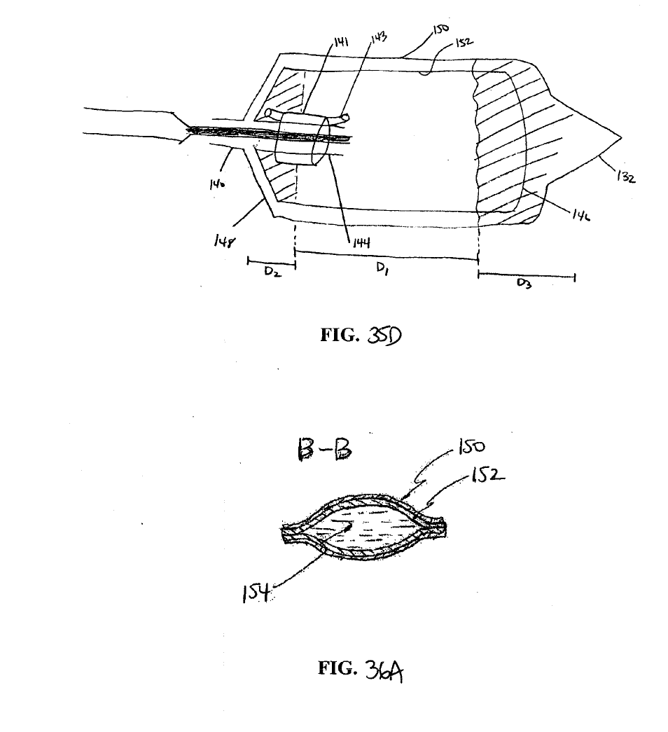

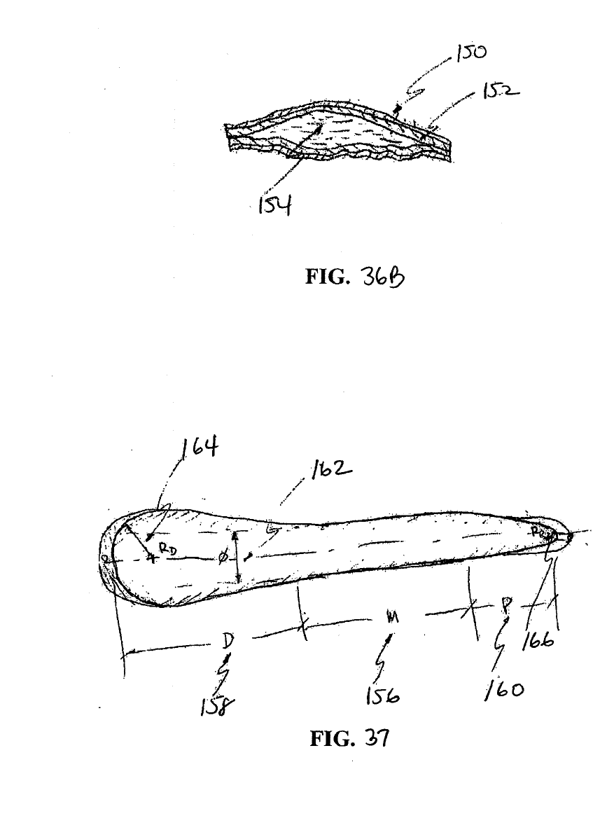

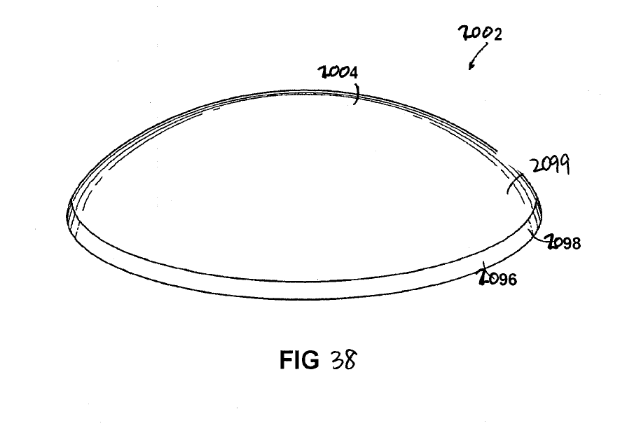

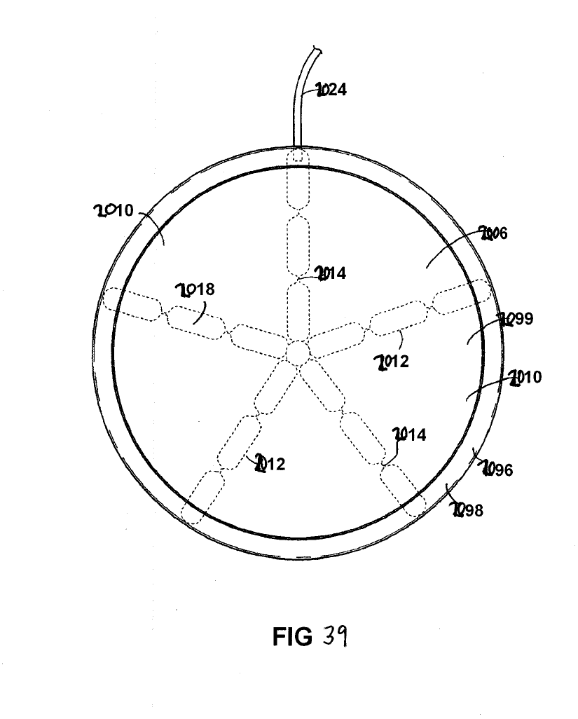

FIG. 34 shows an embodiment of the same implant of FIG. 32 after it has been inflated and after the fill tube has been removed and the self-closing valve assembly has closed to maintain the inflation of the implant. Typically, the implant is inflated with saline, although other materials can be used as well as previously described. The distal taper 146 forms a generally hemispherical or bulb-like shape while the proximal taper 148 forms a more flattened, triangular shape. The advantage of the distal taper shape was described above. The proximal taper 148 inflates to a more flattened shape because the edge of the bond that separates the proximal taper 148 from the proximal tab of the implant follows an arcuate path or other geometry perimeter line that is elongated axially relative to the arcuate path of the edge of the distal tab. As a result, the distance, measured in an axial direction, between the proximal limit 148A and the distal limit 148B of the distal edge of the proximal bond is greater than the corresponding axial distance on the distal end of the implant. The proximal bond edge length is generally at least about 110%, often at least about 150% and can be at least about 200% of the axial length of the distal bond edge. Thus the implant can not inflate as fully in the direction perpendicular to the plane of the tab at a comparable distance from the end of the tab. This design is advantageous in that it more closely matches the required tissue augmentation of this region of the nasolabial area: the subdermal tissue plane in which the implant is optimally placed is at its most superficial in this location relative to the rest of the nasolabial region, from approximately 1 mm to 4 mm deep to the skin, and the depth and extent of the nasolabial crease (which can also be referred to as the nasolabial fold or the nasolabial sulcus) is at its minimum in this location.

The dimensions of a preferred implant are as follows. The inflated axial length may be from about 1-15 cm, preferably about 1.5-8 cm, more preferably about 2-5 cm. The diameter of an inflated implant may be from about 2-10 cm, preferably about 3-8 cm, more preferably about 4-6 cm. The maximal cross-sectional area of an inflated implant may be no more than about 80 cm², preferably no more than about 50 cm², more preferably less than about 30 cm², or even 12 cm² or less. In certain embodiments, it may be desirable to have a uniform diameter and cross-sectional area through the body of the implant. In alternative embodiments, the cross-sectional area may vary along the axial length of the implant, with at a first axial distance from an end there will be a first cross-sectional area, and at a second axial distance from the end there will be a second, different cross-sectional area. This second cross-sectional area may be at least about 110%, 120%, 130%, or more of the first cross-sectional area. These alternative embodiments thus may create an implant that has, for example, a transition such as a uniform taper, progressive curve, accelerated curve, and the like.

FIG. 35A is a cross-sectional view through the implant in the region of the distal taper 146. It is shown first in an embodiment in which the physician has chosen to inflate the implant to its maximum recommended inflation volume, which creates a soft, flaccid implant but is at the highest end of the range of volumes that will create those soft, flaccid characteristics. At this fill-volume, the cross-sectional shape of the implant in the main body section as well as in the distal taper region is close to circular. For example, a preferred maximum fill volume of an implant may be in the range of from about 1-60 cc, preferably from about 5-40 cc, 10-30 cc, or more preferably about 15-25 cc. A preferred flaccid-filled implant may be filled to about, for example, no greater than about 50%, 60%, 70%, 80%, or 90% of the maximum fill volume.

Also seen in FIG. 35A is the dual-layer construction of a preferred embodiment. An outer porous or textured material layer 150, such as expanded polytetrafluoroethylene (ePTFE), porous polyethylene, textured polyurethane or textured silicone contacts the body tissues surrounding the implant. The characteristics of these materials allow for incorporation of the implant into the tissue without encapsulation. This is due to controlled and slight cellular ingrowth that occurs with the properly selected porous or textured material. One such material is ePTFE with a pore size of between 30 and 100 microns, preferably between 50 and 80 microns.

FIG. 35B illustrates an embodiment of an implant with variations in lamination, in which the porous outer material 150 is affixed to the underlying inner elastomeric material 152 in wound or interrupted configurations, for example, helical, bands, stripes, and other discontinuous patterns. These alternative embodiments are beneficial in controlling the total surface area susceptible to tissue ingrowth and reducing capsule formation, such as when it is desired that the implant be removed at a later date. The percentage of the total surface area of the implant which is provided with an outer porous layer can be varied, depending upon the desired clinical result. In general, although 100% coverage may be desired in certain circumstances, the outer porous layer may alternatively cover no more than about 90%, and in some embodiments no more than about 75%, and in further embodiments no more than about 50% of the total surface area of the implant. The configuration of the porous outer layer may also be varied, such that it may be positioned on end zones of the implant, positioned in a central zone on the implant, or distributed throughout such as by a spiral winding, spaced apart transverse rings, checker board pattern, or otherwise.

When the porous layer is provided as a spiral winding, or as a series of transverse circles surrounding the implant, the implant may more readily expand and contract in an axial direction, while maintaining a constant cross sectional profile. In general, the materials utilized for the porous layer (e.g. ePTFE) are less compliant than materials useful for the inner layer. As a consequence, elongation of the implant as a result of over inflation, or compression of one end of the implant will allow axial stretching or expansion of the implant without being constrained by the porous layer. This may among other objectives help provide a natural feel, upon manual palpation of the implant from the surface of the skin.

Within the outer layer 150 is an inner layer 152, which provides a fluid-tight seal and, along with the valve assembly, enables the implant to be inflated and to maintain its inflation. This inner layer 152 is preferably formed from an elastomer, such as dimethylsiloxane (silicone) or polyurethane, and more preferably from an elastomer with a durometer between about 40-00 and 80-A on the Shore hardness scales. This enables the implant to not only be soft and flaccid upon presentation of an initial deforming force but to also have “cushioned stop” when the deformation exceeds the amount that the flaccidity is able to absorb. This inner layer 152 of the implant can also be made of inextensible materials thus relying completely on its flaccidity in order to present mechanical softness; this is particularly suitable in implant locations that are in deeper tissue planes, such as against the periosteum or against bone. The filler 154, is within the inner layer 152.

FIG. 35C is a cross-section of the embodiment of FIG. 35A in which the physician has chosen to inflate the implant to a level below the maximum recommended inflation volume. This creates a markedly flaccid implant that even further conforms to the pocket with the tissue bed created by the physician during implantation. This conformability and flaccidity allows the implant to “blend” with the surrounding tissue in terms of its mechanical properties, and renders it very difficult to palpate. The materials of the implant are generally of an optically clear nature, particularly once in contact with body fluids, and the soft, flaccid shape does not cause it to impart a protrusion on the skin surface; thus the implant is invisible to the eye and very difficult to detect by palpation. Invisibility of detection to the eye and difficulty of palpation are desirable characteristics of implants in cosmetic and reconstructive surgery.

FIG. 35D shows a preferred nasolabial implant and various preferred characteristics. The implant contains both the inner layer 152 made of preferably silicone, and an outer layer 150 which may be made of, for example, ePTFE. There is a proximal bonded zone D2 and distal bonded zone D3 shown shaded, where the inner layer 152 and outer layer 150 are bonded such as by the use of a silicone adhesive. The axial lengths D2 and D3 may be identical, or may be different lengths in different embodiments. Furthermore, there may be a central zone without bonding D1 where the inner layer 152 and outer layer 150 can “slip” and allow for flotation of the inner layer 152 with respect to the outer layer 150. A desirable implant will have compliance matching with the surrounding native tissues to provide a more natural feel and appearance. This unbonded zone D1 allows for elongation of the ePTFE outer layer construct 150 as well as the inner layer 152 which will serve to increase the compliance of the implant. The axial length D1 of the unbonded zone may be anywhere between about 0.5-14 cm, preferably about 1-10 cm, 1.5-6 cm, and often between about 2-4 cm. The length D1 of the unbonded zone may be at least about 10%, 20%, 50%, 67%, 75%, or more of the overall implant length in some embodiments.

The implant is preferably impermeable or minimally permeable to vapor or liquid at physiologic temperatures. Depending on the desired long-term stability of the implant, it may be desirable to add one or more additional layers. This may be desirable, for example, to inhibit permeability at physiologic pressures and temperatures, and thus premature deflation.

FIG. 36A shows a cross-section of the proximal taper 148 of the embodiment of FIG. 34. Note the flattened cross-section that enables the proximal portion to better “feather” into the zone of the nasolabial groove that is close the oral commissure. FIG. 36B shows the effect on the proximal end of inflating the implant of the embodiment of FIG. 36A to a level below the maximum recommended inflation volume, creating a markedly flaccid implant with the same aforementioned advantages.

FIG. 37 illustrates the general shape of an advantageous nasolabial implant in which the distal zone 158 is to the left and the proximal zone 166 to the right, according to one embodiment of the invention. As previously described, it is advantageous for the distal zone 158 to be generally bulbous while the proximal zone 166 is generally a flattened triangular taper. The general size of the implant can be characterized by its diameter 162 which would be measured at the maximum recommended inflation volume when the cross-section would be generally circular and which will characterize the middle zone 156 of the implant. The distal tapered zone 158 can be characterized by its radius of curvature 164 based upon a best fit curve, which can be larger than one-half the diameter 162, thus creating a marked bulbuosity to the distal end zone 158 of the implant. This may be advantageous in patients with particularly deep nasolabial folds, especially in the perialar region. The proximal zone 160 is of a generally triangular shape within the plane of the proximal tab 140 and can be characterized by the length of the tapered zone 160 and the best fit radius of curvature of the interior edge of the seam at the proximal tip 166. The length 160 is, in a preferred embodiment, between one-tenth and six-tenths the length of the middle zone 156. The length of the distal region 158 is, in a preferred embodiment, between one-tenth and six-tenths the length of the middle zone 156.

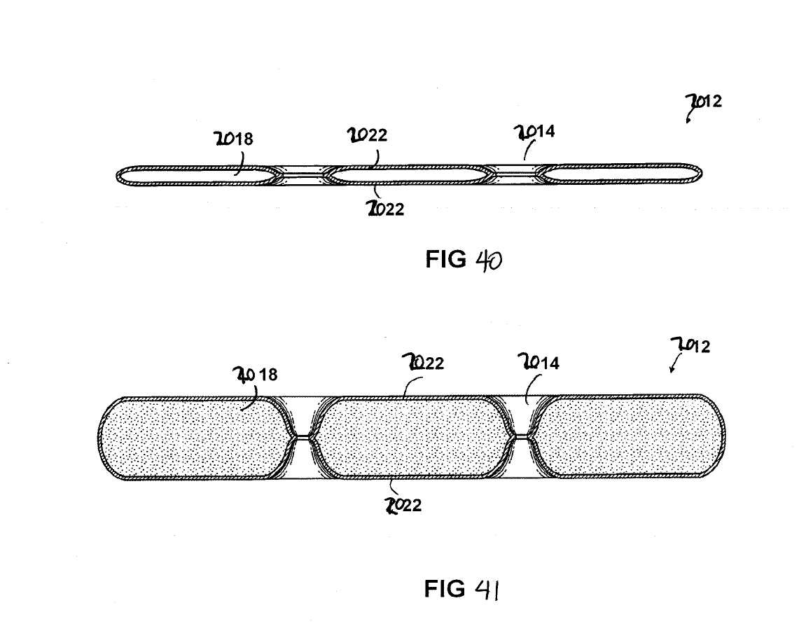

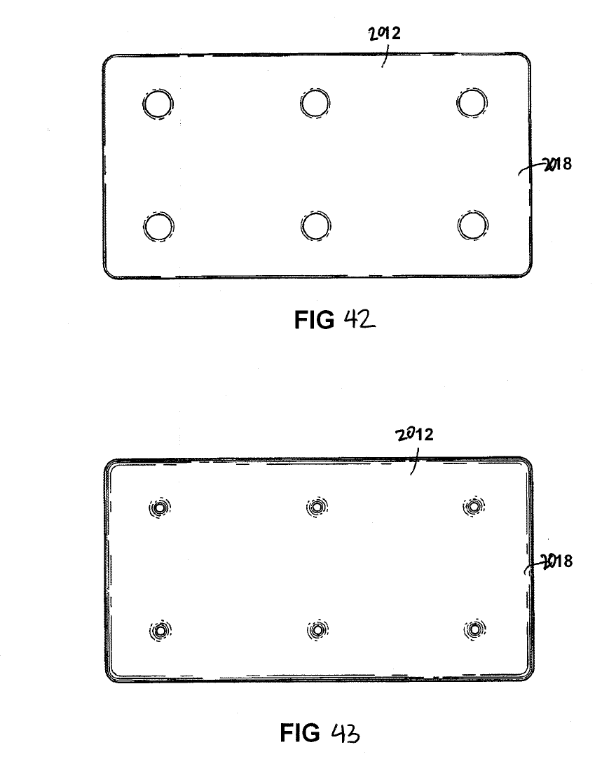

Still further embodiments of implants that can include a porous polymer layer, e.g., ePTFE such as described in connection with FIGS. 1-12 above will now be described. Any of the embodiments disclosed herein may incorporate features, structures and materials of other embodiments as described in FIGS. 1-37 above. Referring to FIGS. 38-43, an implantable prosthesis 2002 is shown. The prosthesis 2002 includes a membrane 2004 which may be made formed in any suitable manner. The membrane 2004 contains a flowable substance 2006 such as silicone gel, saline or any other suitable substance. The flowable substance 2006 may also include elements (not shown), such as beads or spheres, which are suspended in the flowable substance 2006 without departing from the scope of the invention.

The membrane 2004 is divided into a number of chambers 2010 separated by walls 2012. The walls 2012 each have one or more orifices 2014 which have a size which may be adjusted. Changing the size of the orifices 2014 in the walls 2012 alters the flow characteristics of the prosthesis 2002 in that a smaller orifice 2014 will provide a slower flow rate of the flowable substance 2006 between the chambers 2010. The chambers 2010 may also be filled with a substance which further reduces the flow rate of fluid such as an open-cell structure which may be a matrix of material, a sponge, a foam or any other suitable open-cell structure which reduces the flow rate of fluid within the membrane 2004 as described below in connection with other preferred embodiments.

The walls 2012 include an inflatable element 2018 which is inflated or deflated to change the size of the orifice 2014. The inflatable element 2018 may be formed by bonding two sheets of material 2022 together to form the wall 2012. The sheets 2022 are bonded together around the orifices 2014 and a hole is cut to form the orifice 2014. Inflation of the space between the sheets 2022 causes the inflatable element to expand thereby reducing the size of the orifice 2014. A control element 2024 is releasably coupled to the membrane 2004 and is configured to extend out of the patient after the membrane 2004 has been implanted into the patient. The control element 2024 permits the user to change the size of the orifice 2014 after introducing the prosthesis 2002 into the patient. The control element 2024 has a lumen coupled to a source of fluid (not shown) and may be provided with a releasable connection to the membrane 2004 in any suitable manner. Although the control element 2024 is configured to hydraulically alter the size of the orifice 2014, the control element 2024 may accomplish the change in orifice 2014 size using any other method such as mechanical or electrical. For example, the size of the orifice 2014 could be modified using a suture which cinches the orifice 2014 to reduce the size of the orifice 2014.

Referring to FIGS. 44 and 45, another implantable prosthesis 2030 is shown. The prosthesis 2030 includes a membrane 2032 which holds the flowable substance 2006. The membrane 2032 may be filled with an open-cell structure 2034 as described above. The prosthesis 2030 also includes one or more tension elements 2036 which extend between two portions of the wall of the membrane 2032 to help maintain a more stable shape. The tension elements 2036 may extend through a valve 2038 in the prosthesis 2030 which permits the tension element 2036 to slide therethrough while still maintaining a fluid tight seal. The tension element 2036 is coupled to a control element 2040 which may simply be a portion of the tension element 2036 which extends out of the prosthesis 2030. The tension elements 2036 may extend from a posterior wall 2042 to an anterior wall 2044 of the membrane 2032 but may, of course, be coupled to other parts of the membrane 2032 as well.

The control element 2040 is configured to extend out of the patient when the prosthesis 2030 is implanted so that the user may adjust tension on the tension element 2036 after implantation. Tension may be applied to one or more of the tension elements 2036 to create a desirable texture and feel to the prosthesis 2030. After the desired tension has been applied, the control element 2040 may be removed by simply cutting the control element 2040 or releasing the control element 2040 using any other suitable method. A locking element 2043 is coupled to the membrane 2032 which automatically secures the tension element 2036 after tension has been increased with the control element 2040. The control element 2040 may, of course, be manipulated prior to implantation of the prosthesis 2030.

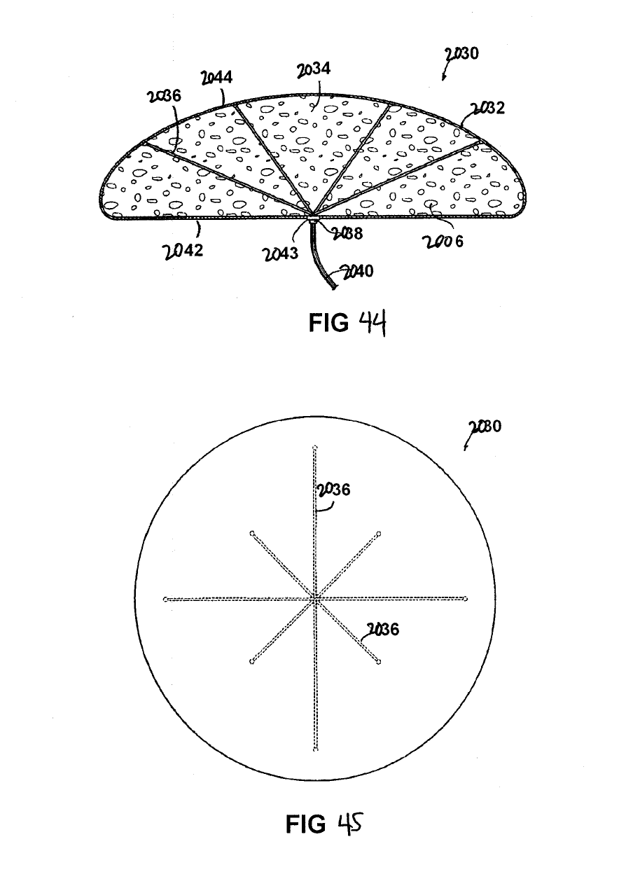

Referring to FIGS. 46 and 47, still another implantable prosthesis 2050 is shown. The prosthesis 2050 includes a membrane 2052 which holds the flowable substance 2006. The membrane 2052 may also contain an open-cell structure 2056 which dampens fluid motion although the invention may be practiced without the open-cell structure 2056. A plurality of tension members 2058 extend through the open-cell structure 2056 and are attached to the membrane 2052 at both ends. The membrane 2052 may have a round posterior wall 2060 which is symmetrical about an axis of symmetry 2062. The tension members 2058 may extend from one side of the membrane 2052 to a diametrically opposed side of the membrane 2052. The tension members 2058 may also be symmetrically arranged relative to the axis of symmetry 2062 and may be coupled together at a junction 2064 so that tension is distributed among the tension members 2058.

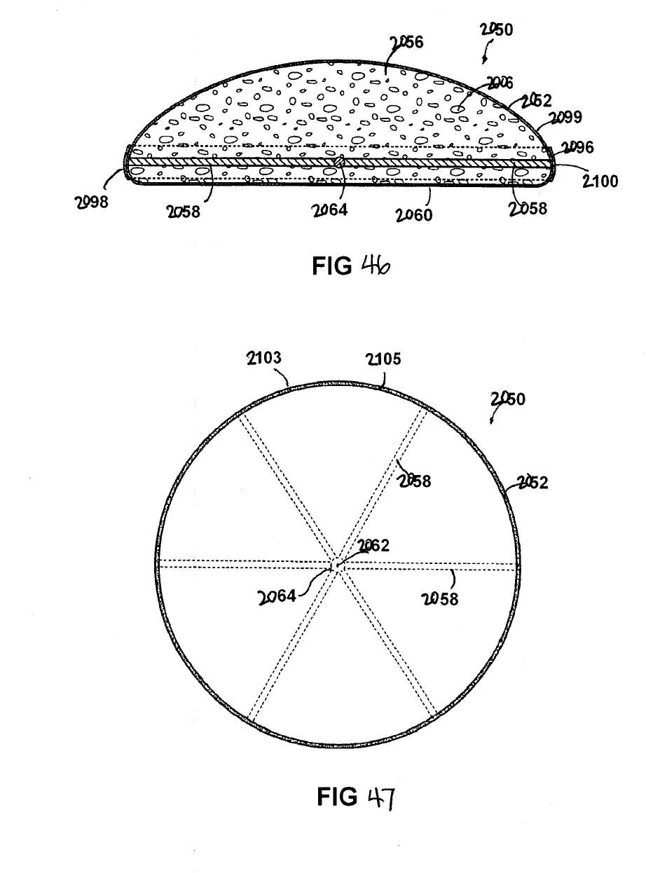

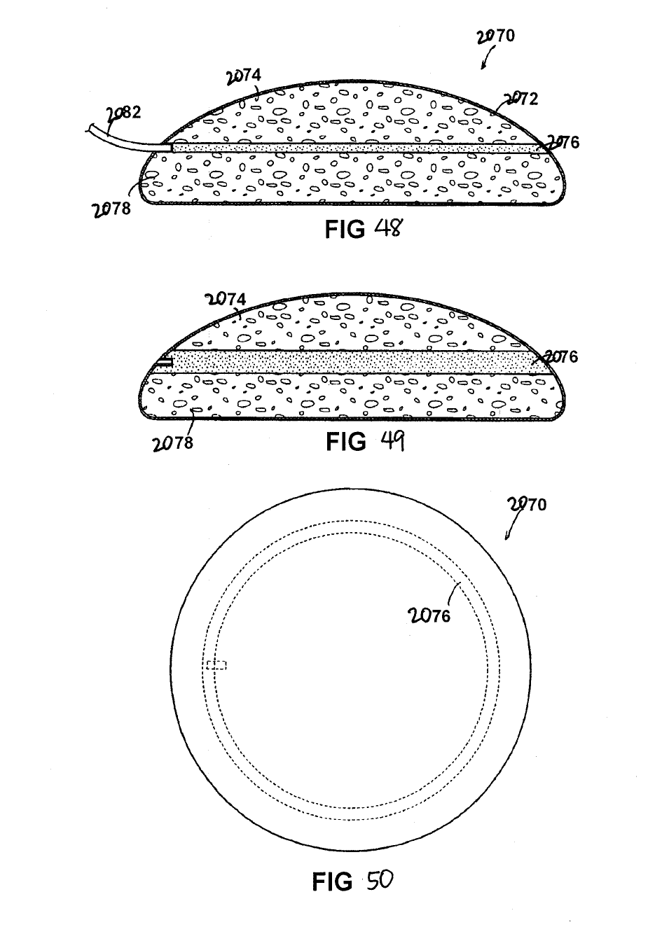

Referring to FIGS. 48-50, yet another implantable prosthesis 2070 is shown. The prosthesis 2070 includes a membrane 2072 having a first chamber 2074, a second chamber 2076 and a third chamber 2078. The chambers 2074, 2076, 2078 may be filled with an open-cell structure 2080. The second chamber 2076 is fluidly isolated from the first and third chambers 2074, 2078 and may be filled using a removable fill line 2082. The second chamber 2076 may be filled or evacuated as desired before or after the prosthesis 2070 has been implanted into a patient. The second chamber 2076 is positioned between the first and third chambers 2074, 2078 and may generally lie in a plane but may be oriented in any other suitable manner. The first and third chambers 2074, 2078 may be fluidly isolated from one another or may be fluidly coupled together.

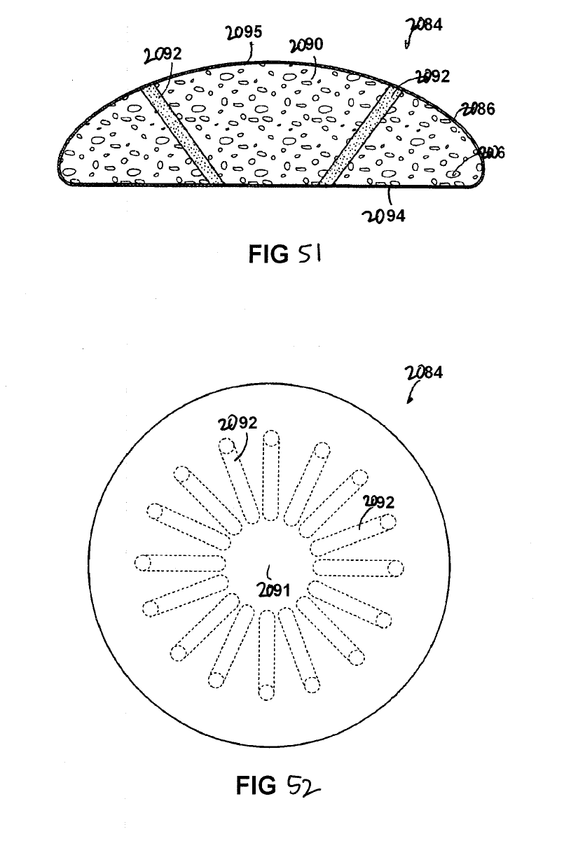

Referring to FIGS. 51 and 52, another implantable prosthesis 2084 is shown. The prosthesis includes a membrane 2086 filled with the flowable substance 2006. The prosthesis 2084 also includes an open-cell structure 2090 which dampens fluid motion and helps to maintain a desired shape. The open-cell structure 2090 includes a plurality of voids 2092 which are substantially larger than an average cell size in the open-cell structure 2090. The membrane 2086 may be symmetrical about an axis of symmetry 2091 which is centrally located relative to a round posterior wall 2094. The round posterior wall 2094 and symmetrical shape permit the user to implant the device without requiring a particular orientation when implanted. The voids 2092 are preferably symmetrically positioned relative to the axis of symmetry 2091. The voids 2092 may be elongate channels 2096 cut into the open-cell structure 2090 which extend from the posterior wall 2094 to an anterior wall 2095 of the prosthesis 2084.

Referring again to FIGS. 38 and 46, a cover 2096 may be used to cover a portion of an outer surface 2099 of the membrane 2004 and may be used with any of the implants described herein. The cover 2096 may be a strip 2098 of expanded PTFE (which can be as described, for example, in connection with FIGS. 1-12 above) which extends over, and essentially parallel to, an area commonly referred to as the waist 2100. The waist 2100 is generally defined as a radially outer portion of the membrane 2052 when the membrane 2052 is supported by the posterior wall 2060 as shown in FIG. 46. The cover 2096 is positioned so that at least 80% of the ePTFE is positioned no more than 1 cm from the waist 2100. Positioning the ePTFE cover 2096 in this manner provides the advantages of ePTFE, such as the promotion of in-growth, without the high cost of covering the entire implant with ePTFE as has been suggested by some prior art devices. Of course, numerous aspects of the present invention may be practiced without the cover 2096 or with the cover 2096 extending around the entire outer surface or a substantial portion thereof without departing from those aspects of the invention.

The cover 2096 may be applied to the membrane 2052 in the following manner when using the strip 2098 of ePTFE. The membrane 2052 is held at two spaced-apart locations 2103, 2105 along the waist 2100 and the membrane 2052 is stretched to increase the space between these locations. The membrane 2052 may be held by a curved work element which supports the curved shape of the membrane when the membrane 2052 is stretched. The strip 2098 is then attached to the membrane at both locations 2103, 2105 and the membrane 2052 is then released to release tension on the membrane 2052. This process may be repeated until the entire waist 2100 is covered by the strip 2098. In one embodiment, the strip 2098 is attached at 6-10 locations around the periphery of the waist 2100.

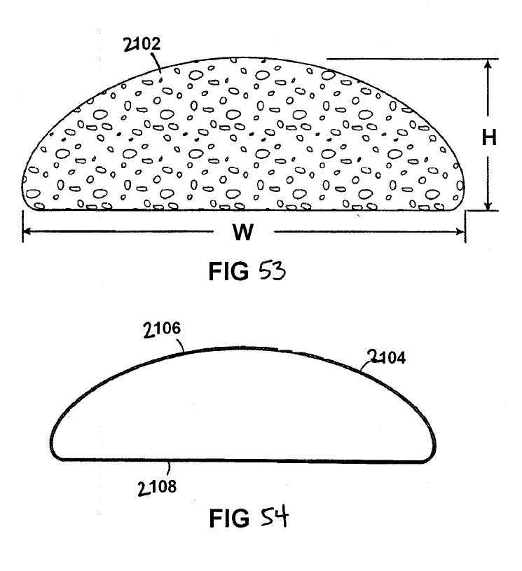

Referring now to FIGS. 53 and 54, still another aspect of the present invention is shown. An open cell structure 2102 is provided which has a natural, unbiased shape which is larger than membrane 2104. The open cell structure 2102 is compressed within the membrane 2104 which holds the open cell structure 2102 in a compressed state. The open cell structure 2102 may occupy a volume when in the natural unbiased shape which is 5% to 20% larger than the volume of the membrane 2104.

The open cell structure 2102 may be larger than the membrane 2104 in all dimensions or may be selectively larger in one or more dimensions. For example, the open cell structure 2100 may have a height H which is 5% to 20% larger than a maximum dimension between an anterior wall 2106 and a posterior wall 2108. The open cell structure 2102 may also have a width W which is 5% to 20% larger than a maximum outer dimension or diameter of the posterior wall 2108.

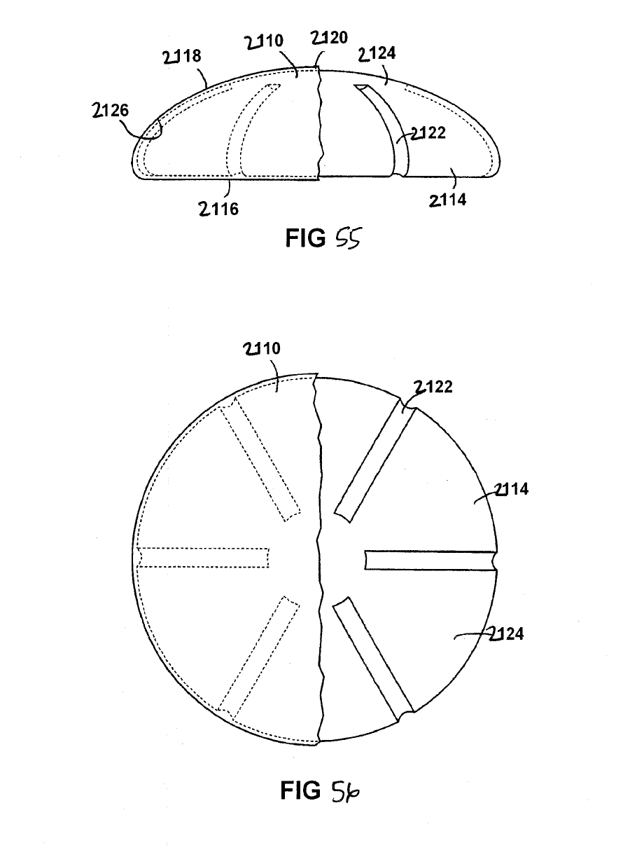

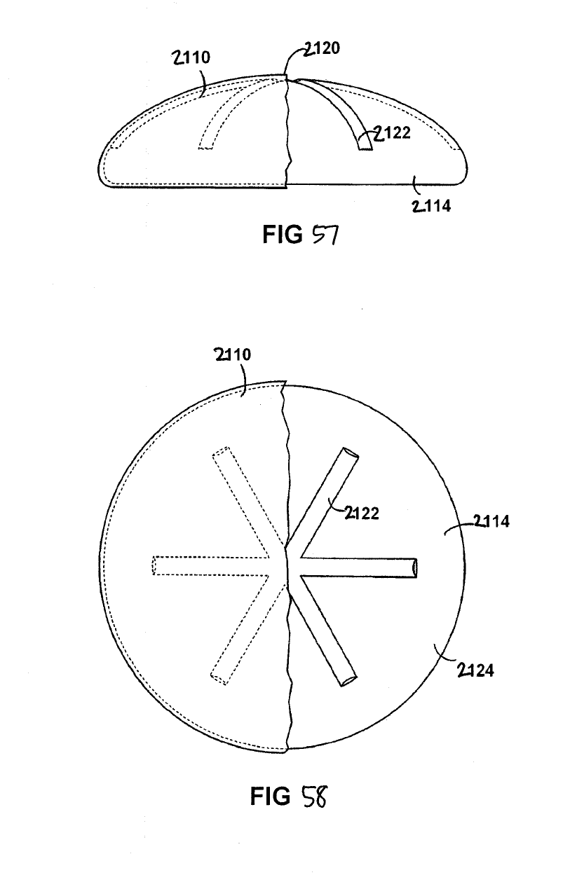

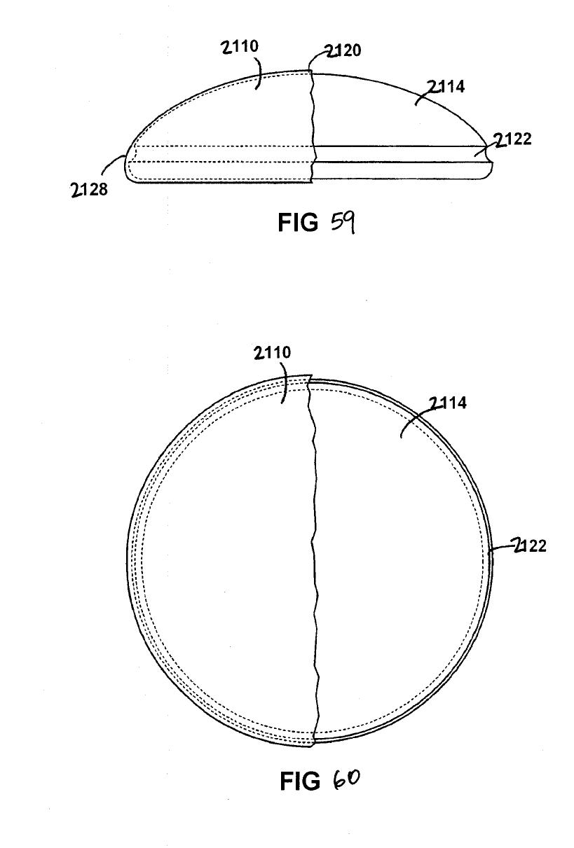

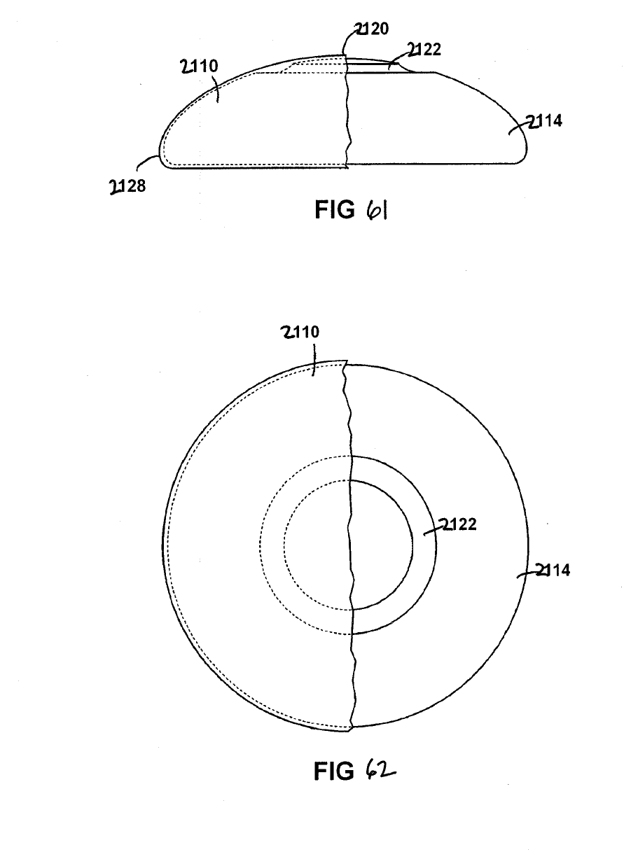

Referring now to FIGS. 55-62, an implantable prosthesis 2109 is shown which has a membrane 2110 and an open cell structure 2114 with channels 2122 formed in an outer surface 2124 of the open cell structure 2114. The membrane 2110 includes a posterior wall 2116 and an anterior wall 2118 having an apex 2120. The channels 2122 may be positioned adjacent to an inner surface 2126 of the membrane 2110 so that the flowable substance can flow in a more unrestricted manner in the channels 2122 than in the open cell structure 2114. The channels 2122 may extend radially relative to the apex 2120 of the membrane 2112 (FIGS. 55-58). The channels 2120 may intersect one another at the inner surface 2126 of the membrane 2112 below the apex 2120 (FIGS. 57 and 58) or may be non-intersecting (FIGS. 55 and 56). Referring to FIGS. 59 and 60, the channel 2122 may also extend circumferentially about the outer surface 2124 of the open cell structure 2114. The channel 2122 may also be positioned adjacent to a waist 2128 of the membrane which is a radially outer portion of the membrane 2110 near the posterior wall 2116 as described above. The channel 2122 may also extend around the apex 2120 of the membrane 2110 at a position nearer to the apex 2120 than to the waist 2128 as shown in FIGS. 61 and 62.

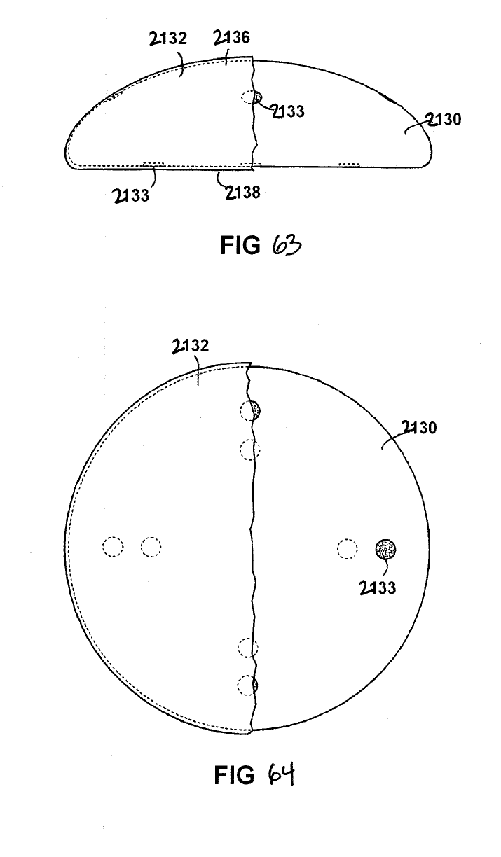

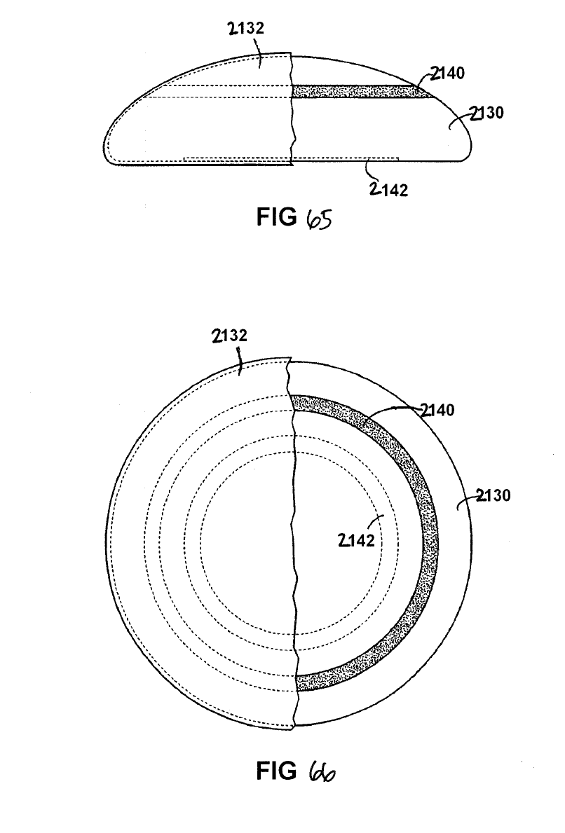

Referring now to FIGS. 63-66, open cell structure 2130 may be attached to membrane 2132 at a selective number of locations which are separated by portions of the open cell structure 2130 which are free to move relative to an inner surface 2134 of the membrane 2132. FIGS. 63 and 64 shows the open cell structure 2130 attached to the membrane 2132 at four spaced apart locations on anterior wall 2136 and posterior wall 2138. FIGS. 65 and 66 show the open cell structure 2130 attached to the membrane 2132 along a strip 2140 on the anterior wall 2136 and along a strip 2142 on the posterior wall 2138. The strip 2136 on the anterior wall 2136 may form a closed loop that encircles the apex of the membrane.

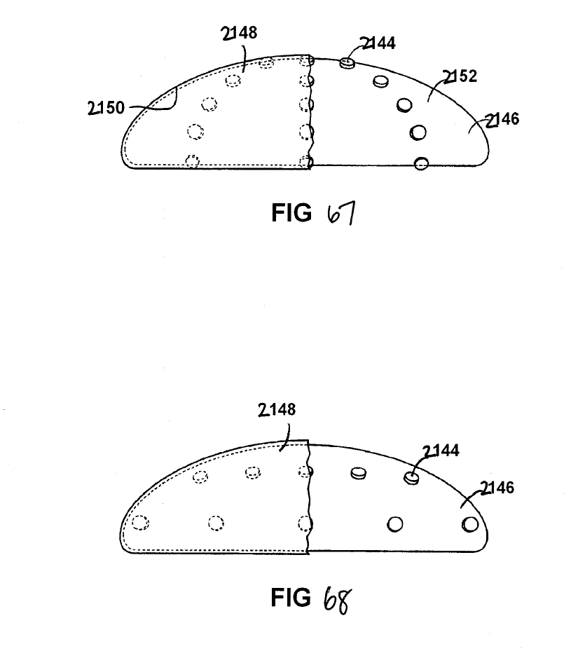

Referring now to FIGS. 67 and 68, spacers 2144 may also be provided between open cell structure 2146 and the membrane 2148. The spacers 2144 create an area between the membrane 2148 and the open cell structure 2146 so that the flowable substance may flow in a less restricted manner in this area as compared to within the open cell structure 2146. The spacers 2144 may be attached to the membrane 2148 or to the open cell structure 2146 and may be integrally formed with either part. When attached to the open cell structure 2146, the spacers 2144 are free to slide against an inner surface 2150 of the membrane 2148. The spacers 2144 may be sized and positioned so that less than 20% of an outer surface 2152 of the open cell structure 2146 is covered by the spacers 2144. Stated another way, at least 80% of the outer surface 2152 of the open cell structure 2146 is free to move relative to the inner surface 2150 of the membrane 2148. The spacers 2144 may be arranged in a radially oriented fashion (FIG. 67) or in a circumferential pattern (FIG. 68) or any other suitable configuration without departing from the scope of the invention.

The present invention has been described in connection with various preferred embodiments and it is understood that modifications and alterations of these embodiments may be accomplished while remaining within the scope of the invention as defined by the claims. For example, the implants may be anatomical implants rather than symmetrical implants without departing from the scope of various aspects of the invention. Furthermore, the various aspects of the invention have been described independently but may, of course, be practiced together and such combinations are expressly incorporated. For example, the spacers 2144 of FIGS. 67 and 68 could be used in combination with the tension elements 2136 of FIGS. 44 and 45.

Although this invention has been disclosed in the context of certain preferred embodiments and examples, it will be understood by those skilled in the art that the present invention extends beyond the specifically disclosed embodiments to other alternative embodiments and/or uses of the invention and modifications and equivalents thereof. Additionally, the skilled artisan will recognize that any of the above-described methods can be carried out using any appropriate apparatus. Further, the disclosure herein of any particular feature in connection with an embodiment can be used in all other disclosed embodiments set forth herein. Thus, it is intended that the scope of the present invention herein disclosed should not be limited by the particular disclosed embodiments described above.

What is claimed is:

1. A method of forming an implant, comprising the steps of:
providing a layer of ePTFE and an inner element, the layer having a width and a length, the layer of ePTFE comprising a plurality of nodes and a plurality of fibrils extending between the nodes;
reducing the length of the ePTFE layer while the ePTFE layer remains a substantially flat sheet; and
attaching the layer of ePTFE to the inner element after the reducing step.

2. The method of claim 1, wherein:
the reducing step is carried out by reducing the length at a plurality of locations along the length of the ePTFE.

3. The method of claim 1, wherein:
the reducing step is carried out by reducing the length of ePTFE in a substantially uniform manner across the length of the ePTFE.

4. The method of claim 1, wherein:
the reducing step is carried out so that the fibrils between the nodes deform under compression to accommodate the reduction in length.

5. The method of claim 1, wherein:
the reducing step is carried out so that a distance between the nodes is decreased along the length while maintaining the nodes in a substantially flat layer.

6. The method of claim 1, wherein:
the reducing step is carried out to deform the layer of ePTFE.

7. The method of claim 1, wherein:
the reducing step is carried out to reduce the length at least 5% while maintaining a substantially smooth surface.

8. The method of claim 1, wherein:
the reducing step is carried out to reduce the length at least 25% while maintaining a substantially smooth surface.

9. The method of claim 1, wherein:
the reducing step is carried out to reduce the length at least 50% while maintaining a substantially smooth surface.

10. The method of claim 1, further comprising the step of:
stretching the layer of ePTFE to increase the width of the ePTFE layer before the reducing step.

11. The method of claim 10, wherein:
the stretching step is carried out to increase the width by at least 5%.

12. The method of claim 10, wherein:
the stretching step is carried out to increase the width by at least 25%.

13. The method of claim 1, wherein:
the providing step is carried out with the inner element being a fluid permeable structure which permits fluid to pass therethrough.

14. The method of claim 13, wherein:
the providing step is carried out with the permeable structure being made of an open cell silicone.

15. A method of forming an implant, comprising the steps of:
providing a layer of ePTFE and an inner element, the layer having a width and a length, the layer of ePTFE comprising a plurality of nodes and a plurality of fibrils extending between the nodes;
reducing the length of the ePTFE layer at least 10% while maintaining a substantially smooth surface of the layer, the reducing step is carried out so that a distance between the nodes is decreased while maintaining the nodes in a substantially flat sheet, the fibrils between the nodes being deformed to accommodate the reduction in length;
stretching the layer of ePTFE to increase the width of the ePTFE layer before the reducing step; and
attaching the layer of ePTFE to the inner element after the reducing step.

16. An implant which may be implanted into a patient, comprising:
a layer of ePTFE having a plurality of fibrils extending between nodes;
an inner element, the layer of ePTFE being attached to the inner element;
wherein the layer of ePTFE has a substantially flat surface, the ePTFE having fibrils which are retained in a deformed, compressed configuration.

17. The implant of claim 16, wherein:
the layer of ePTFE has a length which has been reduced by deforming under compression the fibrils while maintaining the nodes within substantially the same plane.

18. The implant of claim 17, wherein:
the layer of ePTFE has a length which has been reduced by at least 5%.

19. The implant of claim 16, wherein:
the layer of ePTFE has a length which has been reduced by at least 25%.

20. The implant of claim 16, wherein:
the layer of ePTFE has a length which has been reduced by at least 50%.
